SEQUENCE LISTING (1 sequence)

SEQ ID NO: 1 - protein, 785 aa, synthetic construct
  source 1..785: note = human USP1 protein
MPGVIPSESNGLSRGSPSKKNRLSLKFFQKKETKRALDFTDSQENEEKASEYRASEIDQVVPAAQSSPINCEKRENLLPFVGLNNLGNTCYLNSILQVLYFCPGFKSGVKHLFNIISRKKEALKDEANQKDKGNCKEDSLASYELICSLQSLIISVEQLQASFLLNPEKYTDELATQPRRLLNTLRELNPMYEGYLQHDAQEVLQCILGNIQETCQLLKKEEVKNVAELPTKVEEIPHPKEEMNGINSIEMDSMRHSEDFKEKLPKGNGKRKSDTEFGNMKKKVKLSKEHQSLEENQRQTRSKRKATSDTLESPPKIIPKYISENESPRPSQKKSRVKINWLKSATKQPSILSKFCSLGKITTNQGVKGQSKENECDPEEDLGKCESDNTTNGCGLESPGNTVTPVNVNEVKPINKGEEQIGFELVEKLFQGQLVLRTRCLECESLTERREDFQDISVPVQEDELSKVEESSEISPEPKTEMKTLRWAISQFASVERIVGEDKYFCENCHHYTEAERSLLFDKMPEVITIHLKCFAASGLEFDCYGGGLSKINTPLLTPLKLSLEEWSTKPTNDSYGLFAVVMHSGITISSGHYTASVKVTDLNSLELDKGNFVVDQMCEIGKPEPLNEEEARGVVENYNDEEVSIRVGGNTQPSKVLNKKNVEAIGLLGGQKSKADYELYNKASNPDKVASTAFAENRNSETSDTTGTHESDRNKESSDQTGINISGFENKISYVVQSLKEYEGKWLLFDDSEVKVTEEKDFLNSLSPSTSPTSTPYLLFYKKL

(19) United States
(12) Patent Application Publication
(10) Pub. No.: US 2026/0345612 A1
(43) Pub. Date: Nov. 5, 2026

(54) THERAPEUTIC COMBINATIONS COMPRISING UBIQUITIN-SPECIFIC-PROCESSING PROTEASE 1 (USP1) INHIBITORS AND PARP1-SELECTIVE INHIBITORS

(71) Applicant: KSQ Therapeutics, Inc., Lexington, MA (US)

(72) Inventors: Andrew Alistair Wylie, Newton, MA (US); Anne Louise Cadzow, Boston, MA (US); Frank Stegmeier, Acton, MA (US)

(21) Appl. No.: 19/107,693

(22) PCT Filed: Sep. 22, 2023
(86) PCT No.: PCT/US2023/074886

§ 371 Date: Feb. 28, 2025

Related U.S. Application Data
(60) Provisional application No. 63/376,987, filed on Sep. 23, 2022.
(60) Provisional application No. 63/380,742, filed on Oct. 24, 2022.
(60) Provisional application No. 63/496,289, filed on Apr. 14, 2023.

(51) Int. Cl. A61K 31/519; A61K 31/496; A61P 35/00

(52) U.S. Cl. CPC A61K 31/519; A61K 31/496; A61P 35/00

(57) ABSTRACT

The present disclosure provides therapeutic combinations comprising (i) a ubiquitinspecific-processing protease 1 (USP1) inhibitor and (ii) PARP1-selective inhibitor, or a pharmaceutically acceptable salt, hydrate, solvate, amorphous solid, polymorph, or co-crystals thereof, wherein the USP1 inhibitor comprises, or pharmaceutically acceptable salts, hydrates, solvates, amorphous solids, polymorphs, or cocrystals thereof. The present disclosure is also directed to the use of the combinations to inhibit a USP1 and/or PARP1 protein and/or to treat a disorder responsive to the inhibition of USP1 and/or PAR1P proteins and USP1 and/or PARP1 activity. The combinations of the present disclosure are especially useful for treating cancer.

### CROSS REFERENCE TO RELATED APPLICATIONS

This application claims priority to U.S. Provisional Patent Application Nos. 63/376,987, filed Sep. 23, 2022, 63/380,742, filed Oct. 24, 2022, and 63/496,289, filed Apr. 14, 2023, each of which is herein incorporated by reference in its entirety.

### REFERENCE TO SEQUENCE LISTING SUBMITTED ELECTRONICALLY

The content of the electronically submitted sequence listing (Name: 4195_034PC03_Seqlisting_ST26; Size: 2,057 bytes; and Date of Creation: Sep. 21, 2023) is herein incorporated by reference in its entirety.

### BACKGROUND OF THE DISCLOSURE

### Field

The present disclosure provides therapeutic combinations of a ubiquitin-specific-processing protease 1 (USP1) inhibitor and a selective Poly (ADP-ribose) polymerase 1 (PARP1) inhibitor. Methods of treating cancers comprising administering the combinations are also provided.

### Background

Ubiquitin is a small (76 amino acid) protein that is post-transcriptionally attached to target proteins. The consequence of ubiquitination is determined by the number and linkage topology of ubiquitin molecules conjugated to the target protein. For example, proteins exhibiting lysine 48-linked poly-ubiquitin chains are generally targeted to the proteasome for degradation, while mono-ubiquitination or poly-ubiquitin chains linked through other lysines regulate non-proteolytic functions, such as cell cycle regulation, DNA damage repair, transcription, and endocytosis. Ubiquitination is a reversible process, and enzymes called deubiquitinases remove ubiquitin from target proteins.

USP1 is a deubiquitinase that plays a role in DNA damage repair. USP1 interacts with UAF1 (USP1-associated factor 1) to form a complex that is required for the deubiquitinase activity. The USP1/UAF1 complex deubiquitinates mono-ubiquitinated PCNA (proliferating cell nuclear antigen) and mono-ubiquitinated FANCD2 (Fanconi anemia group complementation group D2), which are proteins that play important functions in translesion synthesis (TLS) and the Fanconi anemia (FA) pathway, respectively. The USP1/UAF1 complex also deubiquitinates Fanconi anemia complementation group I (FANCI). These two pathways are essential for repair of DNA damage induced by DNA cross-linking agents, such as cisplatin and mitomycin C (MMC).

The Poly (ADP-ribose) polymerase (PARP) family of enzymes plays roles in DNA repair and genome integrity. PARP is critical for single stranded break repair and base excision repair pathways. A key enzymatic activity is to add ADP-ribose to substrate protein via cleavage of NAD+ and release of nicotinamide. This poly (ADP-ribosyl)ation (“PARylation”) activity is activated by DNA strand breaks, which leads to addition of Par to PARP itself and other DNA repair enzymes. PARP is critical for the recruitment of DNA repair proteins to the damage sites.

Homologous recombination is a DNA repair process crucial for the accurate repair of DNA damage. BRCA1/2 genes, along with other Fanconi anemia pathway genes (e.g., RAD51D, NBN, ATM), are components of homologous recombination-mediated DNA repair. Mutations in the genes encoding homologous recombination factors play roles in the development of certain cancers. PARP inhibitors prevent the repair of DNA single-stranded breaks and promote the conversion of single-stranded breaks to double-stranded breaks, which creates synthetic lethality in cancer cells that lack proficient double-stranded break mechanisms such as homologous recombination.

There remains an unmet medical need for more effective therapies, e.g., combination therapies, for the treatment of cancers.

### BRIEF SUMMARY OF THE DISCLOSURE

Combinations of (i) a ubiquitin-specific-processing protease 1 (USP1) inhibitor or a pharmaceutically acceptable salt, hydrate, solvate, amorphous solid, polymorph, or co-crystal thereof, and (ii) a selective poly ADP-ribose polymerase 1 inhibitor (selective PARP1 inhibitor), or a pharmaceutically acceptable salt, hydrate, solvate, amorphous solid, polymorph, or co-crystal thereof, are provided herein. Also provided herein are methods of treating a subject with cancer using such a combination.

In one aspect, the present disclosure relates to a combination composition comprising (i) a ubiquitin-specific-processing protease 1 (USP1) inhibitor and (ii) PARP1-selective inhibitor, or a pharmaceutically acceptable salt, hydrate, solvate, amorphous solid, polymorph, or co-crystal thereof, wherein the USP1 inhibitor is a compound selected from the group consisting of

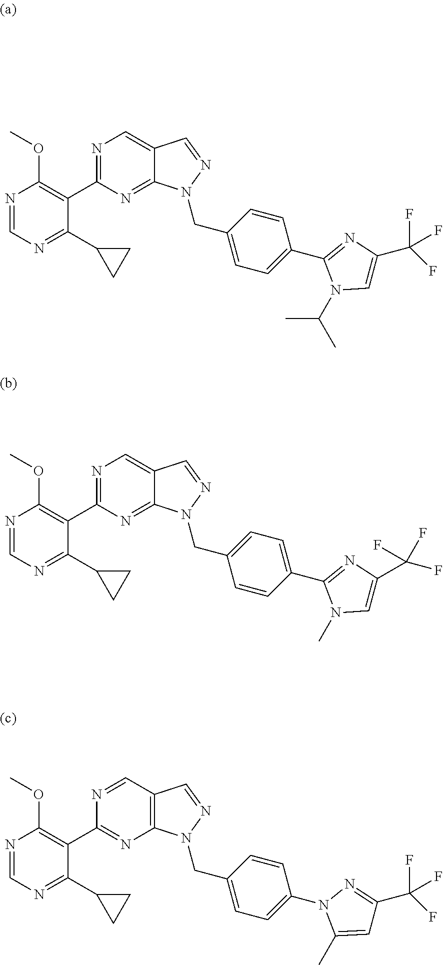 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1 ; COc1ncnc(C2CC2)c1-c1ncc2cnn(Cc3ccc(-n4nc(C(F)(F)F)cc4C)cc3)c2n1]

and pharmaceutically acceptable salts, hydrates, solvates, amorphous solids, polymorphs, or co-crystals thereof. In another aspect, Formula I, Formula II, or Formula III is provided as a co-crystal. In some aspects, Formula I, Formula II, or Formula III is provided as a co-crystal with a pharmaceutically acceptable acid.

In some aspects, the PARP1-selective inhibitor is selected from the group consisting of AZD5305, and pharmaceutically acceptable salts, hydrates, solvates, amorphous solids, polymorphs, or co-crystals thereof.

In one aspect, the USP1 inhibitor is the compound of Formula I, or a pharmaceutically acceptable salt, hydrate, solvate, amorphous solid, polymorph, or co-crystal thereof. In some aspects, Formula I is provided as a co-crystal with a pharmaceutically acceptable acid.

In another aspect, the USP1 inhibitor is the compound of Formula II, or a pharmaceutically acceptable salt, hydrate, solvate, amorphous solid, polymorph, or co-crystal thereof. In some aspects, Formula II is provided as a co-crystal with a pharmaceutically acceptable acid.

In another aspect, the USP1 inhibitor is the compound of Formula III, or a pharmaceutically acceptable salt, hydrate, solvate, amorphous solid, polymorph, or co-crystal thereof. In some aspects, Formula III is provided as a co-crystal with a pharmaceutically acceptable acid.

In another aspect, the USP1 inhibitor is a co-crystal of the compound of Formula I and a second pharmaceutically acceptable compound. In some aspects, a pharmaceutically acceptable co-crystal is formed between a compound of Formula I and a pharmaceutically acceptable acid. In some aspects, the pharmaceutically acceptable acid is selected from the group consisting of benzoic acid, gentisic acid, and salicylic acid.

In another aspect, the USP1 inhibitor is a co-crystal of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine.

In another aspect, the USP1 inhibitor is a co-crystal of gentisic acid and 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine.

In another aspect, the USP1 inhibitor is a gentisate salt of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine.

In one aspect, the present disclosure relates to the use of the combination composition for the manufacture of a medicament for treatment of cancer.

In another aspect, the present disclosure relates to a pharmaceutical combination composition comprising the combination composition and a pharmaceutically acceptable carrier.

In one aspect, the pharmaceutical composition is for use in the treatment of cancer.

In one aspect, the present disclosure relates to a kit comprising the combination composition or the pharmaceutical combination composition, and instructions for administering the combination to a subject having cancer.

In another aspect, the present disclosure relates to a method of treating cancer in a subject comprising administering to the subject (i) USP1 inhibitor and (ii) PARP1-selective inhibitor, or a pharmaceutically acceptable salt, hydrate, solvate, amorphous solid, polymorph, or co-crystal thereof,
wherein the USP1 inhibitor is a compound selected from the group consisting of

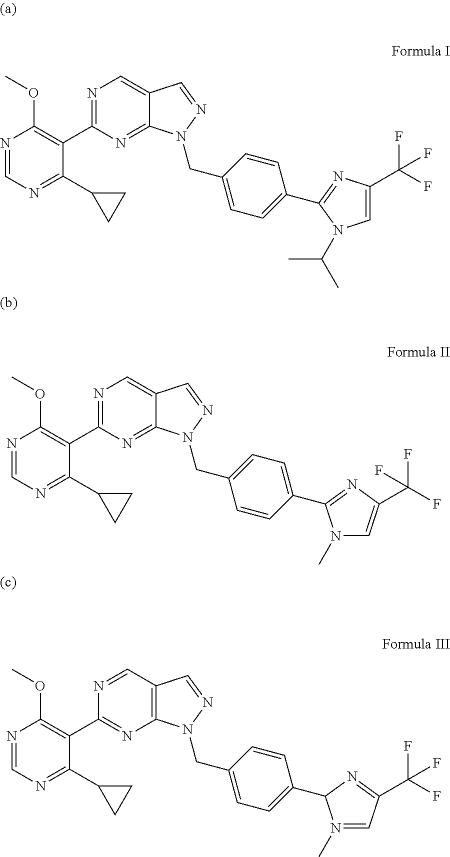

and pharmaceutically acceptable salts, hydrates, solvates, amorphous solids, polymorphs, or co-crystals thereof. In another aspect, Formula I, Formula II, or Formula III is provided as a co-crystal. In some aspects, Formula I, Formula II, or Formula III is provided as a co-crystal with a pharmaceutically acceptable acid.

In one aspect of the method, the PARP1-selective inhibitor is AZD5305 or a pharmaceutically acceptable salt, hydrate, solvate, amorphous solid, polymorph, or co-crystal thereof.

In one aspect of the method, the USP1 inhibitor is the compound of Formula I, or a pharmaceutically acceptable salt, hydrate, solvate, amorphous solid, polymorph, or co-crystal thereof. In some aspects, Formula I is provided as a co-crystal with a pharmaceutically acceptable acid.

In another aspect of the method, the USP1 inhibitor is the compound of Formula II, or a pharmaceutically acceptable salt, hydrate, solvate, amorphous solid, polymorph, or co-crystal thereof. In some aspects, Formula II is provided as a co-crystal with a pharmaceutically acceptable acid.

In another aspect of the method, the USP1 inhibitor is the compound of Formula III, or a pharmaceutically acceptable salt, hydrate, solvate, amorphous solid, polymorph, or co-crystal thereof. In some aspects, Formula III is provided as a co-crystal with a pharmaceutically acceptable acid.

In another aspect, the USP1 inhibitor is a co-crystal of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine.

In another aspect, the USP1 inhibitor is a co-crystal of gentisic acid and 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine.

In another aspect, the USP1 inhibitor is a gentisate salt of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine.

In one aspect of the present disclosure, the administration of the USP1 inhibitor, or said pharmaceutically acceptable salt, hydrate, solvate, amorphous solid, polymorph, or co-crystal thereof, and the selective PARP1 inhibitor, or said pharmaceutically acceptable salt, hydrate, solvate, amorphous solid, polymorph, or co-crystal thereof, provides a synergistic effect.

In one aspect of the present disclosure, the USP1 inhibitor, or said pharmaceutically acceptable salt, hydrate, solvate, amorphous solid, polymorph, or co-crystal thereof, and the PARP1-selective inhibitor, or said pharmaceutically acceptable salt, hydrate, solvate, amorphous solid, polymorph, or co-crystal thereof, are administered in a therapeutically effective amount sufficient to produce one or more therapeutic effects selected from the group consisting of (i) a reduction in tumor size, (ii) an increase in cancer tumor regression rate, (iii) a reduction or inhibition of cancer tumor growth, and (iv) a reduction of the toxicity of a PARP1-selective inhibitor administered as a monotherapy (e.g., due to less PARP1-selective inhibitor being administered). In one aspect of the present disclosure, the USP1 inhibitor, or said pharmaceutically acceptable salt, hydrate, solvate, amorphous solid, polymorph, or co-crystal thereof, and the PARP1-selective inhibitor, or said pharmaceutically acceptable salt, hydrate, solvate, amorphous solid, polymorph, or co-crystal thereof, are administered in a therapeutically effective amount sufficient to produce one or more therapeutic effects selected from the group consisting of (i) a reduction in tumor size, (ii) an increase in cancer tumor regression rate, and (iii) a reduction or inhibition of cancer tumor growth. In one aspect of the present disclosure, the USP1 inhibitor, or said pharmaceutically acceptable salt, hydrate, solvate, amorphous solid, polymorph, or co-crystal thereof, and the PARP1-selective inhibitor, or said pharmaceutically acceptable salt, hydrate, solvate, amorphous solid, polymorph, or co-crystal thereof, are administered in an amount sufficient to reduce the toxicity of a PARP1-selective inhibitor administered as a monotherapy (e.g., due to less PARP1-selective inhibitor being administered).

In one aspect, the USP1 inhibitor, or said pharmaceutically acceptable salt, hydrate, solvate, amorphous solid, polymorph, or co-crystal thereof, and the PARP1-selective inhibitor, or said pharmaceutically acceptable salt, hydrate, solvate, amorphous solid, polymorph, or co-crystal thereof, are administered in a therapeutically effective amount sufficient to delay, reduce, or prevent rebounding (rapid re-growth) of a tumor.

In one aspect, the USP1 inhibitor, or said pharmaceutically acceptable salt, hydrate, solvate, amorphous solid, polymorph, or co-crystal thereof, and the PARP1-selective inhibitor, or said pharmaceutically acceptable salt, hydrate, solvate, amorphous solid, polymorph, or co-crystal thereof, are administered sequentially.

In another aspect, the USP1 inhibitor, or said pharmaceutically acceptable salt, hydrate, solvate, amorphous solid, polymorph, or co-crystal thereof, and the PARP1-selective inhibitor, or said pharmaceutically acceptable salt, hydrate, solvate, amorphous solid, polymorph, or co-crystal thereof, are administered simultaneously.

In one aspect of the present disclosure, the combination is administered to a mammal. In another aspect, the mammal is a human.

In some aspects, the cancer is selected from the group consisting of a hematological cancer, a lymphatic cancer, a solid tumor, a DNA damage repair pathway deficient cancer and a homologous-recombination deficient cancer.

In some aspects, the cancer is an advanced solid tumor.

In some aspects, the cancer is selected from the group consisting of brain cancer, lung cancer, non-small cell lung cancer (NSCLC), colon cancer, bladder cancer, osteosarcoma, ovarian cancer, skin cancer, uterine cancer, peritoneal cancer, and endometrial cancer, and breast cancer.

In some aspects, the cancer is non-small cell lung cancer (NSCLC).

In some aspects, the cancer is colon cancer.

In some aspects, the cancer is bladder cancer.

In some aspects, the cancer is ovarian cancer or breast cancer.

In some aspects, the cancer is ovarian cancer.

In some aspects, the cancer is breast cancer.

In some aspects, the cancer is triple negative breast cancer.

In some aspects, the cancer is selected from the group consisting of bone cancer, including osteosarcoma and chondrosarcoma; brain cancer, including glioma, glioblastoma, astrocytoma, medulloblastoma, and meningioma; soft tissue cancer, including rhabdoid and sarcoma; kidney cancer; bladder cancer; skin cancer, including melanoma; and lung cancer, including non-small cell lung cancer; colon cancer, uterine cancer; nervous system cancer; head and neck cancer; pancreatic cancer; and cervical cancer.

In some aspects, the cancer is a DNA damage repair pathway deficient cancer.

In some aspects, the cancer is a BRCA1 mutant cancer. In some aspects, the BRCA1 mutation is a germline mutation. In some aspects, the BRCA1 mutation is a somatic mutation. In some aspects, the BRCA1 mutation leads to BRCA1 deficiency.

In some aspects, the cancer is a BRCA2 mutant cancer. In some aspects, the BRCA2 mutation is a germline mutation. In some aspects, the BRCA2 mutation is a somatic mutation. In some aspects, the BRCA2 mutation leads to BRCA2 deficiency.

In some aspects, the cancer is a BRCA1 mutant cancer and a BRCA2 mutant cancer.

In some aspects, the cancer is a BRCA1 deficient cancer.

In some aspects, the cancer is a BRCA2 deficient cancer.

In some aspects, the cancer is a BRCA1 deficient cancer and a BRCA2 deficient cancer.

In some aspects, the cancer is a PARP inhibitor refractory or resistant cancer. In some aspects, the cancer is a PARP inhibitor resistant or refractory BRCA1, BRCA2, or BRCA1 and BRCA2 mutant cancer. In some aspects, the cancer is a PARP inhibitor resistant or refractory BRCA1, BRCA2, or BRCA1 and BRCA2-deficient cancer.

In some aspects, the cancer is an Olaparib refractory or resistant cancer. In some aspects, the cancer is an Olaparib resistant or refractory BRCA1, BRCA2, or BRCA1 and BRCA2 mutant cancer. In some aspects, the cancer is an Olaparib resistant resistant or refractory BRCA1, BRCA2, or BRCA1 and BRCA2-deficient cancer.

In some aspects, the cancer is an AZD-5305 refractory or resistant cancer. In some aspects, the cancer is an AZD-5305 resistant or refractory BRCA1, BRCA2, or BRCA1 and BRCA2 mutant cancer. In some aspects, the cancer is an AZD-5305 resistant resistant or refractory BRCA1, BRCA2, or BRCA1 and BRCA2-deficient cancer.

In some aspects, the cancer has a mutation in the gene encoding ataxia telangiectasia mutated (ATM) protein kinase. In some aspects, the ATM mutation is a germline mutation. In some aspects, the ATM mutation is a somatic mutation. In some aspects, the cancer is an ATM-deficient cancer.

In some aspects, the cancer comprises cancer cells with a mutation in a gene encoding p53. In some aspects, the mutation in a gene encoding p53 is a germline mutation. In some aspects, the mutation in a gene encoding p53 is a somatic mutation. In some aspects, the cancer comprises cancer cells with a loss of function mutation in a gene encoding p53.

In some aspects, the cancer has a mutation in the gene encoding at least two of p53, BRCA1, BRCA2, and ATM.

In some aspects, the cancer was previously treated with a platinum therapy. In some aspects, the cancer is platinum resistant or platinum refractory. In some aspects, the platinum therapy is selected from the group consisting of cisplatin, carboplatin, oxaliplatin, and combinations thereof.

In another aspect, the present disclosure relates to a method of treating a USP1 protein mediated disorder and/or a PARP protein mediated disorder comprising administering to a subject in need thereof a USP1 inhibitor of Formula I, Formula II, or Formula III, or a pharmaceutically acceptable salt, hydrate, solvate, amorphous solid, polymorph, or co-crystal thereof, and a PARP1-selective inhibitor, or a pharmaceutically acceptable salt, hydrate, solvate, amorphous solid, polymorph, or co-crystal thereof, in an effective amount to treat the USP1 protein mediated disorder and/or the PARP protein mediated disorder.

In another aspect, the present disclosure relates to a method of inhibiting a USP1 protein and/or a PARP protein comprising contacting a USP1 protein and/or a PARP protein with a USP1 inhibitor of Formula I, Formula II, or Formula III, or a pharmaceutically acceptable salt, hydrate, solvate, amorphous solid, polymorph, or co-crystal thereof, and a PARP1-selective inhibitor, or a pharmaceutically acceptable salt, hydrate, solvate, amorphous solid, polymorph, or co-crystal thereof.

In some aspects, the contacting occurs in vitro.

In some aspects, the contacting occurs in vivo.

In one aspect of the method, the PARP1-selective inhibitor is AZD5305 or a pharmaceutically acceptable salt, hydrate, solvate, amorphous solid, polymorph, or co-crystal thereof.

In one aspect of the method, the USP1 inhibitor is the compound of Formula I, or a pharmaceutically acceptable salt, hydrate, solvate, amorphous solid, polymorph, or co-crystal thereof. In some aspects, Formula I is provided as a co-crystal with a pharmaceutically acceptable acid.

In another aspect of the method, the USP1 inhibitor is the compound of Formula II, or a pharmaceutically acceptable salt, hydrate, solvate, amorphous solid, polymorph, or co-crystal thereof. In some aspects, Formula II is provided as a co-crystal with a pharmaceutically acceptable acid.

In another aspect of the method, the USP1 inhibitor is the compound of Formula III, or a pharmaceutically acceptable salt, hydrate, solvate, amorphous solid, polymorph, or co-crystal thereof. In some aspects, Formula III is provided as a co-crystal with a pharmaceutically acceptable acid.

In another aspect, the USP1 inhibitor is a co-crystal of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine.

In another aspect, the USP1 inhibitor is a co-crystal of gentisic acid and 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine.

In another aspect, the USP1 inhibitor is a gentisate salt of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine.

In one aspect, the present disclosure relates to the use of the combinations for the manufacture of a medicament for treatment of cancer.

In another aspect, the present disclosure relates to a pharmaceutical composition comprising the combinations and a pharmaceutically acceptable carrier.

In one aspect, the pharmaceutical composition is for use in the treatment of cancer.

In one aspect, the present disclosure relates to a kit comprising the combination or the pharmaceutical composition, and instructions for administering the combination to a subject having cancer.

Additional aspects and advantages of the disclosure will be set forth, in part, in the description that follows, and will flow from the description, or can be learned by practice of the disclosure. The aspects and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### BRIEF DESCRIPTION OF THE FIGURES

FIG. 1A shows that a USP1 inhibitor of Formula I enhances antitumor activity of AZD5305 in a Breast PDX Model.

FIG. 1B shows the efficacy of a combination of a USP1 inhibitor of Formula I and AZD5305 in mice with tumors with resistance to PARP inhibitors.

FIG. 1C shows the tolerability of a combination of a USP1 inhibitor of Formula I and AZD5305.

FIG. 2 shows an XRPD pattern corresponding to crystalline Form 2.

FIG. 3 shows a DSC and TGA thermogram corresponding to crystalline Form 2.

FIG. 4 shows a colony formation assay (CFU) of UWB1.289 parental cells treated with Formula I and/or AZD5305.

FIG. 5 shows a colony formation assay (CFU) of UWB1.289 BRCA1 overexpressing cells treated with Formula I and/or AZD5305.

### DETAILED DESCRIPTION OF THE DISCLOSURE

One aspect of the present disclosure is based on the use of a combination of a ubiquitin-specific-processing protease 1 (USP1) protein inhibitor and a PARP1-selective inhibitor. The combinations are useful for inhibiting a USP1 protein and/or a PARP protein and for treating diseases, disorders, or conditions, e.g., cancer, that are responsive to inhibition of a USP1 protein and/or a PARP protein.

In some aspects, the combination of a USP1 inhibitor and a PARP1-selective inhibitor provide a synergistic effect.

In some aspects, the USP1 inhibitor and the PARP1-selective inhibitor are in therapeutically effective amounts sufficient to produce a therapeutic effect comprising: (i) a reduction in size of a tumor, (ii) an increase in cancer tumor regression rate, (iii) a reduction or inhibition of cancer tumor growth, and/or (iv) a reduction of the toxicity of a PARP1-selective inhibitor administered as a monotherapy (e.g., due to less PARP1-selective inhibitor being administered). In some aspects, the USP1 inhibitor and the PARP1-selective inhibitor can delay, reduce, or prevent rebounding (rapid re-growth) of a tumor.

The tolerability (lack of toxicity) of combinations provided herein is particular surprising given that other combinations with the PARP inhibitor Olaparib have not been well-tolerated. See, e.g., Samol, J., et al., Invest. New Drugs, 30:1493-500 (2012) (“Further development of olaparib and topotecan in combination was not explored due to dose-limiting hematological AEs and the resulting sub-therapeutic MTD.”).

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

It is understood that aspects described herein include “consisting” and/or “consisting essentially of” aspects. As used herein, the singular form “a,” “an,” and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about,” as used herein, includes the recited number ±10%. Thus, “about 10” means 9 to 11. As is understood by one skilled in the art, reference to “about” a value or parameter herein includes (and describes) instances that are directed to that value or parameter per se. For example, description referring to “about X” includes description of “X.”

The present disclosure encompasses the preparation and use of salts of the USP1 inhibitors and PARP1-selective inhibitors, including non-toxic pharmaceutically acceptable salts. Examples of pharmaceutically acceptable addition salts include inorganic and organic acid addition salts and basic salts. Pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparaginate, glutamate and the like. The term “pharmaceutically acceptable salt” as used herein, refers to any salt, e.g., obtained by reaction with an acid or a base, of a USP1 inhibitor or PARP1-selective inhibitor of the disclosure that is physiologically tolerated in the target patient (e.g., a mammal, e.g., a human).

Acid addition salts can be formed by mixing a solution of the particular USP1 inhibitor or PARP1-selective inhibitor with a solution of a pharmaceutically acceptable non-toxic acid such as hydrochloric acid, fumaric acid, maleic acid, succinic acid, acetic acid, citric acid, tartaric acid, carbonic acid, phosphoric acid, oxalic acid, dichloroacetic acid, or the like. Basic salts can be formed by mixing a solution of the USP1 inhibitor or PARP1-selective inhibitor of the present disclosure with a solution of a pharmaceutically acceptable non-toxic base such as sodium hydroxide, potassium hydroxide, choline hydroxide, sodium carbonate and the like.

In some aspects of the disclosure, a pharmaceutically acceptable salt is formed between a compound of Formula I, Formula II, or Formula III and a pharmaceutically acceptable acid. In some aspects, the pharmaceutically acceptable salt of a compound of Formula I, Formula II, or Formula III is a crystalline form.

In some aspects, the pharmaceutically acceptable acid is selected from the group consisting of 1-hydroxy-2-naphthoic acid, 4-aminosalicylic acid, ascorbic acid, adipic acid, L-aspartic acid, benzene sulfonic acid, benzoic acid, trans-cinnamic acid, citric acid, ethanedisulfonic acid, fumaric acid, galactaric acid, gentisic acid gluconic acid, D-glucuronic acid, glutamic acid, glutaric acid, glycolic acid, hexanoic acid, hippuric acid, hydrobromic acid, hydrochloric acid, lactic acid, maleic acid, L-malic acid, malonic acid, R-mandelic acid, methanesulfonic acid, mucic acid, naphthalene sulfonic acid, nicotinic acid, oxalic acid, palmitic acid, p-toluene sulfonic acid, phosphoric acid, propionic acid, saccharin, salicylic acid, stearic acid, succinic acid, sulfuric acid, L-tartaric acid, vanillic acid, vanillin, ethyl maltol, gallic acid, gallic acid ethyl ester, 4-hydroxybenzoic acid, 4-hydroxybenzoic acid methyl ester, 3,4,5-trihydroxybenzoic acid, nicotinamide, L-proline, and D-sorbitol. In some aspects, the pharmaceutically acceptable acid is selected from the group consisting of 1-hydroxy-2-naphthoic acid, 4-aminosalicylic acid, ascorbic acid, adipic acid, L-aspartic acid, benzene sulfonic acid, benzoic acid, trans-cinnamic acid, citric acid, ethanedisulfonic acid, fumaric acid, galactaric acid, gentisic acid, gallic acid, gluconic acid, D-glucuronic acid, glutamic acid, glutaric acid, glycolic acid, hexanoic acid, hippuric acid, hydrobromic acid, hydrochloric acid, lactic acid, maleic acid, L-malic acid, malonic acid, R-mandelic acid, methanesulfonic acid, mucic acid, naphthalene sulfonic acid, nicotinic acid, oxalic acid, palmitic acid, p-toluene sulfonic acid, phosphoric acid, propionic acid, saccharin, salicylic acid, stearic acid, succinic acid, sulfuric acid, L-tartaric acid, vanillic acid, vanillin, ethyl maltol, gallic acid, gallic acid ethyl ester, 4-hydroxybenzoic acid, 4-hydroxybenzoic acid methyl ester, 3,4,5-trihydroxybenzoic acid, nicotinamide, L-proline, and D-sorbitol. In some aspects, the pharmaceutically acceptable acid is selected from the group consisting of hydrochloric acid, hydrobromic acid, ethanedisulfonic acid, methanesulfonic acid, gentisic acid, benzoic acid, salicylic acid, and gallic acid. In some aspects, the pharmaceutically acceptable acid is selected from the group consisting of hydrochloric acid, hydrobromic acid, ethanedisulfonic acid, methanesulfonic acid, and gentisic acid. In some aspects, the pharmaceutically acceptable acid is a benzoic acid derivative. In some aspects, the pharmaceutically acceptable acid is a benzoic acid derivative substituted with one or more hydroxy groups. In some aspects, the pharmaceutically acceptable acid is a benzoic acid derivative substituted with one hydroxy group. In some aspects, the benzoic acid derivative substituted with one hydroxy group is salicylic acid. In some aspects, the pharmaceutically acceptable acid is a benzoic acid derivative substituted with two hydroxy groups. In some aspects, the benzoic acid derivative substituted with two hydroxy groups is gentisic acid. In some aspects, the pharmaceutically acceptable acid is a benzoic acid derivative substituted with three hydroxy groups. In some aspects, the benzoic acid derivative substituted with three hydroxy groups is gallic acid. In some aspects, the benzoic acid derivative is selected from the group consisting of salicylic acid, gentisic acid, and gallic acid. In some aspects, the pharmaceutically acceptable acid is hydrochloric acid. In some aspects, the pharmaceutically acceptable acid is hydrobromic acid. In some aspects, the pharmaceutically acceptable acid is ethanedisulfonic acid. In some aspects, the pharmaceutically acceptable acid is methanesulfonic acid. In some aspects, the pharmaceutically acceptable acid is gentisic acid. In some aspects, the pharmaceutically acceptable acid is benzoic acid. In some aspects, the pharmaceutically acceptable acid is salicylic acid. In some aspects, the pharmaceutically acceptable acid is gallic acid.

The present disclosure encompasses the preparation and use of solvates of the USP1 inhibitor and/or PARP1-selective inhibitor. Solvates typically do not significantly alter the physiological activity or toxicity of the compounds, and as such may function as pharmacological equivalents. The term “solvate” as used herein is a combination, physical association and/or solvation of a USP1 inhibitor or PARP1-selective inhibitor of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. USP1 inhibitors or PARP1-selective inhibitors of the disclosure can be present as solvated forms with a pharmaceutically acceptable solvent, such as water, methanol, ethanol, and the like, and it is intended that the disclosure includes both solvated and unsolvated forms of the USP1 inhibitor and/or PARP1-selective inhibitor of the disclosure. One type of solvate is a hydrate. A “hydrate” relates to a particular subgroup of solvates where the solvent molecule is water. An “anhydrate” as applied to a compound refers to a solid state wherein the compound contains no structural water within the crystal lattice. Solvates typically can function as pharmacological equivalents. Preparation of solvates is known in the art. See, for example, M. Caira et al, J. Pharmaceut. Sci., 93 (3): 601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5 (1): Article 12 (2004), and A. L. Bingham et al., Chem. Commun. 603-604 (2001). A typical, non-limiting, process of preparing a solvate would involve dissolving a USP1 inhibitor or PARP1-selective inhibitor of the disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

In some aspects of the disclosure, the USP1 inhibitor and/or PARP1-selective inhibitor is deuterated. In some aspects, the USP1 inhibitor and/or PARP1-selective inhibitor are partially or completely deuterated, i.e., one or more hydrogen atoms are replaced with deuterium atoms.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

In the context of cancer, the term “treating” includes, but is not limited to, inhibiting growth of cancer cells, inhibiting replication of cancer cells, lessening of overall tumor burden, and delaying, halting, or slowing tumor growth, progression, or metastasis.

As used herein, “delaying” means to defer, hinder, slow, retard, stabilize, suppress and/or postpone development or progression of the disease (such as cancer). This delay can be of varying lengths of time, depending on the history of the disease and/or individual being treated.

A “therapeutically effective amount” of a substance can vary according to factors such as the disease state, age, sex, and weight of the individual, and the ability of the substance to elicit a desired response in the individual. A therapeutically effective amount is also one in which any toxic or detrimental effects of the substance are outweighed by the therapeutically beneficial effects. A therapeutically effective amount can be delivered in one or more administrations. A therapeutically effective amount refers to an amount effective, at dosages and for periods of time necessary, to achieve the desired therapeutic effect.

The terms “combination,” “therapeutic combination,” “combination composition,” “combination therapy” or “pharmaceutical combination,” as used herein, can include a fixed combination in one dosage unit form, separate dosage units or a kit of parts or instructions for the combined administration where the USP1 inhibitor and the PARP1-selective inhibitor can be administered independently at the same time or separately within time intervals. A combined pharmaceutical composition can be adapted for simultaneous, separate, or sequential administration.

The combination therapy can provide “synergy” and prove “synergistic,” i.e., the effect achieved when the active ingredients used together is greater than the sum of the effects that results from using the compounds separately. A synergistic effect can include a significantly reduced effective dose for the combination of the two active ingredients as compared to the effective dose of each active ingredient when administered separately. A synergistic effect can also include a reduction in toxicity for the combination of the two active ingredients as compared to the toxicity of each active ingredient when administered separately. A synergistic effect can also be an effect that cannot be achieved by administration of any of the active ingredients as single agents. The synergistic effect can include, but is not limited to, an effect of treating cancer by reducing tumor size, inhibiting tumor growth, or increasing survival of the subject. The synergistic effect can also include reducing cancer cell viability, inducing cancer cell death, and inhibiting or delaying cancer cell growth. A synergistic effect can be attained, for example, when the active ingredients are: (1) co-formulated and administered or delivered simultaneously in a combined, unit dosage formulation; (2) delivered serially, by alternation, or in parallel as separate formulations; or (3) by some other regimen. When delivered in alternation therapy, a synergistic effect can be attained when the compounds are administered or delivered sequentially.

A determination of a synergistic interaction between a USP1 inhibitor and a PARP1-selective inhibitor can be based on the results obtained from the assays described herein. For example, combination effects can be evaluated using the Bliss independence model. Bliss scores quantify degree of potentiation from single agents, and a Bliss score >0 suggests greater than simple additivity. In some aspects, a Bliss score greater than 10 indicates strong synergy. In some aspects, a score of 6 or greater indicates synergy. In some aspects, the Bliss score is about 6, about 7, about 8, about 9, about 10, about 11, about 12, about 13, about 14, about 15, about 16, about 17, about 18, about 19, about 20 or about 25.

As used herein, a “homologous recombination deficiency score” or “HRD score” means an algorithmic assessment of three measures of tumor genomic instability, i.e., loss of heterozygosity, telomeric allelic imbalance and large-scale state transitions.

The terms “administer,” “administering,” “administration,” and the like refer to methods that can be used to enable delivery of the therapeutic agent to the desired site of biological action. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa. Administration of two or more therapeutic agents includes simultaneous (concurrent) and consecutive administration in any order.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

The term “container” means any receptacle and closure therefore suitable for storing, shipping, dispensing, and/or handling a pharmaceutical product.

The term “insert” or “package insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

The term “disease” or “condition” or “disorder” as used herein refers to a condition where treatment is needed and/or desired and denotes disturbances and/or anomalies that as a rule are regarded as being pathological conditions or functions, and that can manifest themselves in the form of particular signs, symptoms, and/or malfunctions. As demonstrated below, combinations of the USP1 inhibitors and PARP1-selective inhibitors of the present disclosure can be used in treating diseases and conditions, such as proliferative diseases, wherein inhibition of USP1 and/or PARP1 provides a benefit.

The terms “polypeptide” and “protein” are used interchangeably to refer to a polymer of amino acid residues and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

“USP1” and “ubiquitin-specific-processing protease 1” as used herein refer to any native polypeptide or USP1-encoding polynucleotide. The term “USP1” encompasses “full-length,” unprocessed USP1 polypeptide as well as any forms of USP1 that result from processing within the cell (e.g., removal of the signal peptide). The term also encompasses naturally occurring variants of USP1, e.g., those encoded by splice variants and allelic variants. The USP1 polypeptides described herein can be isolated from a variety of sources, such as from human tissue types or from another source, or prepared by recombinant or synthetic methods. Human USP1 sequences are known and include, for example, the sequences publicly available as UniProt No. 094782 (including isoforms). As used herein, the term “human USP1 protein” refers to USP1 protein comprising the amino acid sequence as set forth in SEQ ID NO:1:

(SEQ ID NO: 1)
MPGVIPSESNGLSRGSPSKKNRLSLKFFQKKETKRALDFTDSQENEE
KASEYRASEIDQVVPAAQSSPINCEKRENLLPFVGLNNLGNTCYLNS
ILQVLYFCPGFKSGVKHLFNIISRKKEALKDEANQKDKGNCKEDSLA
SYELICSLQSLIISVEQLQASFLLNPEKYTDELATQPRRLLNTLREL
NPMYEGYLQHDAQEVLQCILGNIQETCQLLKKEEVKNVAELPTKVEE
IPHPKEEMNGINSIEMDSMRHSEDFKEKLPKGNGKRKSDTEFGNMKK
KVKLSKEHQSLEENQRQTRSKRKATSDTLESPPKIIPKYISENESPR
PSQKKSRVKINWLKSATKQPSILSKFCSLGKITTNQGVKGQSKENEC
DPEEDLGKCESDNTTNGCGLESPGNTVTPVNVNEVKPINKGEEQIGF
ELVEKLFQGQLVLRTRCLECESLTERREDFQDISVPVQEDELSKVEE
SSEISPEPKTEMKTLRWAISQFASVERIVGEDKYFCENCHHYTEAER
SLLFDKMPEVITIHLKCFAASGLEFDCYGGGLSKINTPLLTPLKLSL
EEWSTKPTNDSYGLFAVVMHSGITISSGHYTASVKVTDLNSLELDKG
NFVVDQMCEIGKPEPLNEEEARGVVENYNDEEVSIRVGGNTQPSKVL
NKKNVEAIGLLGGQKSKADYELYNKASNPDKVASTAFAENRNSETSD
TTGTHESDRNKESSDQTGINISGFENKISYVVQSLKEYEGKWLLFDD
SEVKVTEEKDFLNSLSPSTSPTSTPYLLFYKKL.

USP1 is a deubiquitinating enzyme that acts as part of a complex with UAF1. USP1's “deubiquitinase activity” includes its ability to deubiquitinate as part of the USP1-UAF1 complex.

“PARP” or “PARP protein” as used herein refers to one or more of the Poly (ADP-ribose) polymerase family of enzymes. The family includes enzymes that have the ability to catalyze the transfer of ADP-ribose to target proteins (poly ADP-ribosylation). There are at least 18 members of the PARP family that are encoded by different genes, and share homology in a conserved catalytic domain, including PARP1, PARP2 and PARP3.

As used herein the term “PARP1-selective inhibitor” refers to an inhibitor that has selectively for PARP1 over PARP2. In some aspects, a PARP1-selective inhibitor has 500-fold selectivity for PARP1 over PARP2. One example of a PARP1-selective inhibitor is AZD5305.

The term “specifically binds” is well understood in the art, and methods to determine such specific binding are also well known in the art. A molecule is said to exhibit “specific binding” or “preferential binding” if it reacts or associates more frequently, more rapidly, with greater duration and/or with greater affinity with a particular protein or domain of a protein than it does with alternative proteins or domains. It should be understood that a molecule that specifically or preferentially binds to a first protein or domain may or may not specifically or preferentially bind to a second protein or domain. As such, “specific binding” or “preferential binding” does not necessarily require (although it can include) exclusive binding. Generally, but not necessarily, reference to binding means preferential binding. For example, a USP1 inhibitor that specifically binds to USP1, UAF1, and/or the USP1-UAF1 complex may not bind to other deubiquitinases, other USP proteins, or other UAF1 complexes (e.g., USP46-UAF1) or may bind to other deubiquitinases, other USP proteins, or other UAF1 complexes (e.g., USP46-UAF1) with a reduced affinity as compared to binding to USP1.

The terms “reduction” or “reduce” or “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some aspects, by “reduce” or “inhibit” is meant the ability to cause an overall decrease of 20% or greater. In some aspects, by “reduce” or “inhibit” is meant the ability to cause an overall decrease of 50% or greater. In some aspects, by “reduce” or “inhibit” is meant the ability to cause an overall decrease of 75%, 85%, 90%, 95%, or greater. In some aspects, the amount noted above is inhibited or decreased over a period of time, relative to a control over the same period of time.

In some aspects, inhibiting USP1 proteins is the inhibition of one or more activities or functions of USP1 proteins. It should be appreciated that the activity or function of the one or more USP1 proteins may be inhibited in vitro or in vivo. Non-limiting examples of activities and functions of USP1 include deubiquitinase activity and formation of a complex with UAF1 and are described herein. Exemplary levels of inhibition of the activity of one or more USP1 proteins include at least 10% inhibition, at least 20% inhibition, at least 30% inhibition, at least 40% inhibition, at least 50% inhibition, at least 60% inhibition, at least 70% inhibition, at least 80% inhibition, at least 90% inhibition, and up to 100% inhibition.

In some aspects, inhibiting PARP proteins is the inhibition of one or more activities or functions of PARP proteins. It should be appreciated that the activity or function of the one or more PARP proteins may be inhibited in vitro or in vivo. Non-limiting examples of activities and functions of PARP are described herein. Exemplary levels of inhibition of the activity of one or more PARP proteins include at least 10% inhibition, at least 20% inhibition, at least 30% inhibition, at least 40% inhibition, at least 50% inhibition, at least 60% inhibition, at least 70% inhibition, at least 80% inhibition, at least 90% inhibition, and up to 100% inhibition.

The terms “individual” or “subject” are used interchangeably herein to refer to an animal, for example, a mammal, such as a human. In some instances, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some instances, the subject to receive the treatment can be a patient, designating the fact that the subject has been identified as having a disorder of relevance to the treatment, or being at particular risk of contracting the disorder.

As used herein, the terms “cancer” and “tumor” refer to or describe the physiological condition in mammals in which a population of cells are characterized by unregulated cell growth. The terms encompass solid and hematological/lymphatic cancers. Examples of cancer include but are not limited to, DNA damage repair pathway deficient cancers. Additional examples of cancer include, but are not limited to, ovarian cancer, breast cancer (including triple negative breast cancer), non-small cell lung cancer (NSCLC), and osteosarcoma. The cancer can be BRCA1 or BRCA2 wild type. The cancer can also be BRCA1 or BRCA2 mutant. The cancer can further be a PARP inhibitor resistant or refractory cancer, or a PARP inhibitor resistant or refractory BRCA1 or BRCA2-mutant cancer.

As used herein, the term “loss of function” mutation refers to a mutation that results in the absence of a gene, decreased expression of a gene, or the production of a gene product (e.g. protein) having decreased activity or no activity. Loss of function mutations include for example, missense mutations, nucleotide insertions, nucleotide deletions, and gene deletions. Loss of function mutations also include dominant negative mutations. Thus, cancer cells with a loss of function mutation in a gene encoding p53 include cancer cells that contain missense mutations in a gene encoding p53 as well as cancer cells that lack a gene encoding p53.

### USP1 Inhibitors

USP1 inhibitors have been disclosed, for example, in WO2020/132269 and WO2022/094096, each of which is herein incorporated by reference in its entirety.

In some aspects, the ubiquitin-specific-processing protease 1 (USP1) inhibitor of the disclosure comprises a compound of

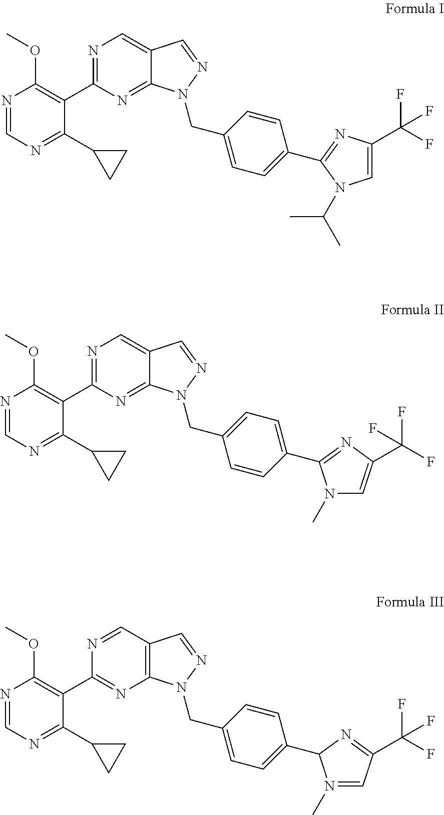

or a pharmaceutically acceptable salt, hydrate, solvate, amorphous solid, polymorph, or co-crystal thereof. In some aspects, the ubiquitin-specific-processing protease 1 (USP1) inhibitor is a compound selected from the group consisting of:

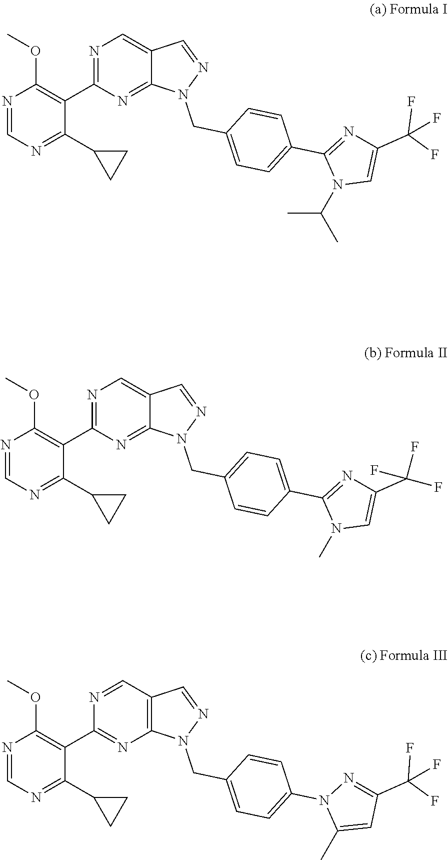 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1 ; COc1ncnc(C2CC2)c1-c1ncc2cnn(Cc3ccc(-n4nc(C(F)(F)F)cc4C)cc3)c2n1]

and a pharmaceutically acceptable salt, hydrate, solvate, amorphous solid, polymorph, or co-crystal thereof.

The chemical name for the USP1 inhibitor of Formula I is 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine, as described in U.S. Publication No. 2021/0115049. This compound is also referred to as Formula I herein.

The chemical name for the USP1 inhibitor of Formula II is 6-(4-cyclopropyl-6-methoxypyrimidin-5-yl)-1-(4-(1-methyl-4-(trifluoromethyl)-1H-imidazol-2-yl)benzyl)-1H-pyrazolo[3,4-d]pyrimidine, as described in U.S. Publication No. 2021/0115049. This compound is also referred to as Formula II herein.

The chemical name for the USP1 inhibitor of Formula III is 6-(4-cyclopropyl-6-methoxypyrimidin-5-yl)-1-(4-(5-methyl-3-(trifluoromethyl)-1H-pyrazol-1-yl)benzyl)-1H-pyrazolo[3,4-d]pyrimidine, as described in U.S. Publication No. 2021/0115049, which is herein incorporated by reference in its entirety. This compound is also referred to as Formula III herein.

In some aspects, a USP1 inhibitor for the uses and methods provided herein is a solid state form that is a pharmaceutically acceptable salt of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Formula I). In some aspects, the pharmaceutically acceptable salt is formed between 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Formula I) and a pharmaceutically acceptable acid. In some aspects, the pharmaceutically acceptable acid is gentisic acid.

In some aspects, the gentisate salt of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Formula I gentisate) is an amorphous form. In some aspects, the amorphous form is substantially free of other polymorphic forms. In some aspects, the amorphous form has a polymorphic purity of at least 70%, or at least 75%, or at least 80%, or at least 85%, or at least 86%, or at least 87%, or at least 88%, or at least 89%, or at least 90%, or at least 91%, or at least 92%, or at least 93%, or at least 94%, or at least 95%, or at least 96%, or at least 97%, or at least 98%, or at least 99%.

In another aspect, the USP1 inhibitor for the uses and methods provided herein is a solid state form that is a co-crystal of a pharmaceutically acceptable acid and 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Formula I). In some aspects, the co-crystal is formed between 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Formula I) and a pharmaceutically acceptable acid selected from the group consisting of 1-hydroxy-2-naphthoic acid, 4-aminosalicylic acid, ascorbic acid, adipic acid, L-aspartic acid, benzene sulfonic acid, benzoic acid, trans-cinnamic acid, citric acid, ethanedisulfonic acid, fumaric acid, galactaric acid, gentisic acid, gluconic acid, D-glucuronic acid, glutamic acid, glutaric acid, glycolic acid, hexanoic acid, hippuric acid, hydrobromic acid, hydrochloric acid, lactic acid, maleic acid, L-malic acid, malonic acid, R-mandelic acid, methanesulfonic acid, mucic acid, naphthalene sulfonic acid, nicotinic acid, oxalic acid, palmitic acid, p-toluene sulfonic acid, phosphoric acid, propionic acid, saccharin, salicylic acid, stearic acid, succinic acid, sulfuric acid, L-tartaric acid, vanillic acid, vanillin, ethyl maltol, gallic acid, gallic acid ethyl ester, 4-hydroxybenzoic acid, 4-hydroxybenzoic acid methyl ester, 3,4,5-trihydroxybenzoic acid, nicotinamide, L-proline, and D-sorbitol. In some aspects, the pharmaceutically acceptable acid is selected from the group consisting of gentisic acid, benzoic acid, salicylic acid, and gallic acid. In some aspects, the pharmaceutically acceptable acid is gentisic acid. Co-crystals of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine are disclosed, for example, in WO2022/094096, which is herein incorporated by reference in its entirety.

In some aspects, a co-crystal of gentisic acid and 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Formula I gentisic acid co-crystal) is an amorphous form. In some aspects, the amorphous form is substantially free of other polymorphic forms. In some aspects, the amorphous form has a polymorphic purity of at least 70%, or at least 75%, or at least 80%, or at least 85%, or at least 86%, or at least 87%, or at least 88%, or at least 89%, or at least 90%, or at least 91%, or at least 92%, or at least 93%, or at least 94%, or at least 95%, or at least 96%, or at least 97%, or at least 98%, or at least 99%.

In some aspects, the gentisate salt of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Formula I gentisate) is a crystalline form.

In some aspects, the pharmaceutically acceptable co-crystal of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Formula I) is a gentisic acid co-crystal.

In some aspects, the pharmaceutically acceptable co-crystal of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine of Formula I is a benzoic acid co-crystal.

In some aspects, the pharmaceutically acceptable co-crystal of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine of Formula I is a salicylic acid co-crystal.

In some aspects, the gentisic acid co-crystal of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Formula I gentisic acid co-crystal) is crystalline Form 2 (Formula I gentisic acid co-crystal, Form 2 or “Form 2”). Formula I gentisic acid co-crystal, Form 2 is characterized by an XRPD pattern having peaks at 16.6±0.2, 18.7±0.2, and 22.5±0.2 degrees two theta.

In some aspects, crystalline Form 2 is an anhydrate.

In some aspects, the melting point of crystalline Form 2 is from about 184° C. to about 190° C. In some aspects, the melting point of crystalline Form 2 is from about 186° C. to about 188° C. In some aspects, the melting point of crystalline Form 2 is about 187° C.

In some aspects, crystalline Form 2 is characterized by an XRPD pattern having peaks at 16.6±0.2, 18.7±0.2, and 22.5±0.2 degrees two theta when measured by Cu Kα radiation. In another aspect, crystalline Form 2 is characterized by an XRPD pattern having peaks at 16.6±0.2, 18.7±0.2, 22.3±0.2, and 22.5±0.2 degrees two theta when measured by Cu Kα radiation. In some aspects, crystalline Form 2 is characterized by an XRPD pattern having peaks at 16.6±0.2, 18.7±0.2, 22.3±0.2, 22.5±0.2, and 26.0±0.2 degrees two theta when measured by Cu Kα radiation. In some aspects, crystalline Form 2 is characterized by an XRPD pattern having peaks at 16.6±0.2, 18.7±0.2, 20.8±0.2, 22.3±0.2, 22.5±0.2, and 26.0±0.2 degrees two theta when measured by Cu Kα radiation.

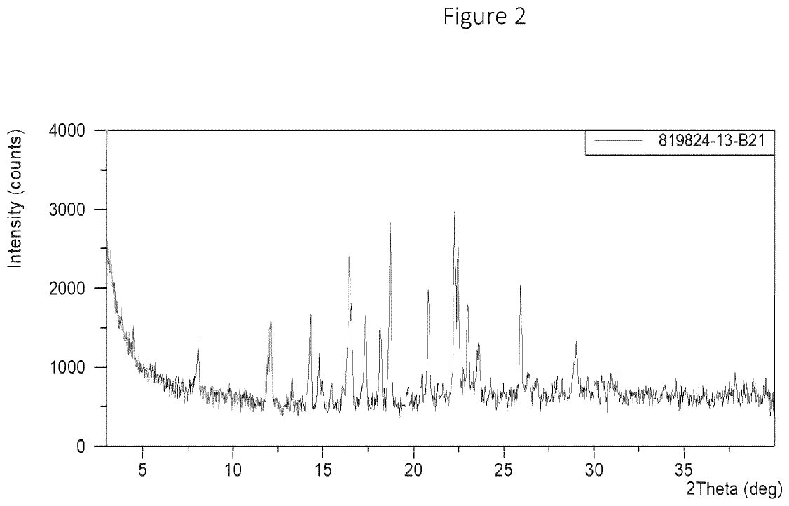

In some aspects, crystalline Form 2 is characterized by an XRPD pattern substantially as shown in FIG. 2. In some aspects, crystalline Form 2 is characterized by three or more, four or more, five or more, or six or more XRPD peaks listed in Table 1 (a and b) below:

TABLE 1a
Select XRPD Peaks for Crystalline Form 2.

| Pos. [°2Th.] | d-spacing [Å] | Rel. Int. [%] |
|---|---|---|
| 8.06 | 10.97 | 21.93 |
| 12.12 | 7.30 | 23.48 |
| 13.30 | 6.66 | 9.94 |
| 14.31 | 6.19 | 37.35 |
| 14.95 | 5.93 | 21.37 |
| 15.47 | 5.73 | 12.00 |
| 16.42 | 5.40 | 38.25 |
| 16.58 | 5.35 | 59.48 |
| 17.35 | 5.11 | 18.11 |
| 18.18 | 4.88 | 30.28 |
| 18.72 | 4.74 | 61.10 |
| 19.68 | 4.51 | 5.01 |
| 20.40 | 4.35 | 6.62 |
| 20.80 | 4.27 | 37.19 |
| 22.29 | 3.99 | 95.77 |
| 22.49 | 3.95 | 100.00 |
| 23.00 | 3.87 | 21.16 |
| 23.65 | 3.76 | 19.85 |
| 25.96 | 3.43 | 38.67 |
| 26.40 | 3.38 | 7.96 |
| 26.88 | 3.32 | 8.15 |
| 28.95 | 3.08 | 8.97 |
| 30.06 | 2.97 | 6.51 |

TABLE 1b
XRPD Peaks for Crystalline Form 2

| Pos. [°2Th.] | d-spacing [Å] | Rel. Int. [%] |
|---|---|---|
| 8.06 | 10.97 | 21.93 |
| 12.12 | 7.30 | 23.48 |
| 13.30 | 6.66 | 9.94 |
| 14.31 | 6.19 | 37.35 |
| 14.95 | 5.93 | 21.37 |
| 15.47 | 5.73 | 12.00 |
| 16.42 | 5.40 | 38.25 |
| 16.58 | 5.35 | 59.48 |
| 17.35 | 5.11 | 18.11 |
| 18.18 | 4.88 | 30.28 |
| 18.72 | 4.74 | 61.10 |
| 19.68 | 4.51 | 5.01 |
| 20.40 | 4.35 | 6.62 |
| 20.80 | 4.27 | 37.19 |
| 22.29 | 3.99 | 95.77 |
| 22.49 | 3.95 | 100.00 |
| 23.00 | 3.87 | 21.16 |
| 23.65 | 3.76 | 19.85 |
| 25.96 | 3.43 | 38.67 |
| 26.40 | 3.38 | 7.96 |
| 26.88 | 3.32 | 8.15 |
| 28.95 | 3.08 | 8.97 |
| 30.06 | 2.97 | 6.51 |
| 31.12 | 2.87 | 2.70 |
| 34.18 | 2.62 | 1.65 |

In some aspects, crystalline Form 2 is characterized by an endothermic peak at from about 181° C. to about 191° C., or from about 183° C. to about 189° C., or from about 185° C. to about 187° C., as determined by DSC. In some aspects, crystalline Form 2 is characterized by an endothermic peak at about 186.0° C., as determined by DSC.

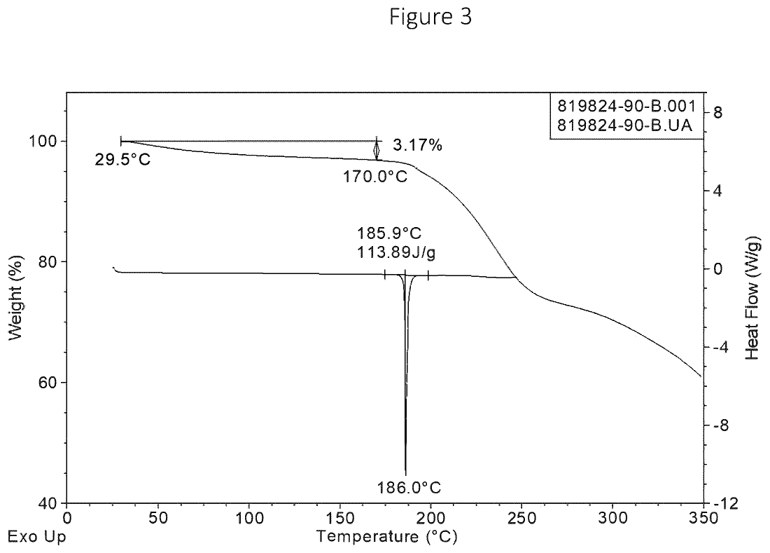

In some aspects, crystalline Form 2 is characterized by a DSC profile substantially as shown in FIG. 3.

In some aspects, crystalline Form 2 is characterized by from an about 2.5 wt % to an about 3.5 wt % loss between room temperature and about 170° C. In some aspects, crystalline Form 2 is characterized by from an about 3.0 wt % to an about 3.4 wt % loss between room temperature and about 170° C. In some aspects, crystalline Form 2 is characterized by an about 3.17 wt % loss between room temperature and about 170° C.

In some aspects, crystalline Form 2 is characterized by a TGA profile substantially as shown in FIG. 3.

In some aspects, crystalline Form 2 is characterized by at least two of the following: a) an XRPD pattern as shown in FIG. 2; b) a DSC profile as shown in FIG. 3; or c) a TGA profile as shown in FIG. 3.

In some aspects, crystalline Form 2 has a unit cell that indexes as monoclinic.

In some aspects, crystalline Form 2 has a unit cell with an a value of about 11.113 Å, a b value of about 12.356 Å, and a c value of about 24.048 Å. In some aspects, Form 2 has a unit cell with a volume of about 3223.93 Å³.

The unit cell parameters for crystalline Form 2 shown in Table 2.

TABLE 2
Unit Cell Parameters for Crystalline Form 2.

| Crystal System | Monoclinic |
|---|---|
| a [Å] | 11.113 |
| b [Å] | 12.356 |
| c [Å] | 24.048 |
| α [deg] | 90 |
| β [deg] | 102.48 |
| γ [deg] | 90 |
| Volume [Å³] | 3223.93 |
| Z CalMolated density [g/cm³] | 4, 1.419 |
| Crystal Size [mm³] | 0.31 × 0.15 × 0.11 |
| Space Group(s) | P2_{1/c} |

In some aspects, crystalline Form 2 is substantially free of other polymorphic forms. In some aspects, crystalline Form 2 has a polymorphic purity of at least 70%, or at least 75%, or at least 80%, or at least 85%, or at least 86%, or at least 87%, or at least 88%, or at least 89%, or at least 90%, or at least 91%, or at least 92%, or at least 93%, or at least 94%, or at least 95%, or at least 96%, or at least 97%, or at least 98%, or at least 99%.

In some aspects, the a USP1 inhibitor comprises a mixture comprising crystalline Form 2 and a second solid state form of Formula I. In some aspects, the second solid state form of Formula I is crystalline Form A. In some aspects, a USP1 inhibitor comprises a mixture comprising a majority of crystalline Form 2 as compared to other solid state forms of Formula I. Suitable solid state forms of Formula I are described in WO2022/094096, which is herein incorporated by reference in its entirety.

In another aspect, the USP1 inhibitor is Formula II, or a pharmaceutically acceptable salt, hydrate, solvate, amorphous solid, polymorph, or co-crystal thereof. In another aspect, the USP1 inhibitor is a solid state form of Formula II, or a pharmaceutically acceptable salt, hydrate, solvate, amorphous solid, polymorph, or co-crystal thereof.

In some aspects, a USP1 inhibitor for the uses and methods provided herein is a solid state form that is a co-crystal of a pharmaceutically acceptable acid and 6-(4-cyclopropyl-6-methoxypyrimidin-5-yl)-1-(4-(1-methyl-4-(trifluoromethyl)-1H-imidazol-2-yl)benzyl)-1H-pyrazolo[3,4-d]pyrimidine Formula II. In some aspects, a pharmaceutically acceptable salt is formed between 6-(4-cyclopropyl-6-methoxypyrimidin-5-yl)-1-(4-(1-methyl-4-(trifluoromethyl)-1H-imidazol-2-yl)benzyl)-1H-pyrazolo[3,4-d]pyrimidine Formula II and a pharmaceutically acceptable acid.

Suitable solid state forms of Formula II are described in WO2022/094096, which is herein incorporated by reference in its entirety.

In another aspect, the USP1 inhibitor is Formula III, or a pharmaceutically acceptable salt, hydrate, solvate, amorphous solid, polymorph, or co-crystal thereof. In another aspect, the USP1 inhibitor is Formula III, or a pharmaceutically acceptable salt, hydrate, solvate, amorphous solid, polymorph, or co-crystal thereof.

In some aspects, a USP1 inhibitor for the uses and methods provided herein is a solid state form that is a co-crystal of a pharmaceutically acceptable acid and 6-(4-cyclopropyl-6-methoxypyrimidin-5-yl)-1-(4-(5-methyl-3-(trifluoromethyl)-1H-pyrazol-1-yl)benzyl)-1H-pyrazolo[3,4-d]pyrimidine Formula III. In some aspects, a pharmaceutically acceptable salt is formed between 6-(4-cyclopropyl-6-methoxypyrimidin-5-yl)-1-(4-(5-methyl-3-(trifluoromethyl)-1H-pyrazol-1-yl)benzyl)-1H-pyrazolo[3,4-d]pyrimidine Formula III and a pharmaceutically acceptable acid.

Suitable solid state forms of Formula III are described in WO2022/094096, which is herein incorporated by reference in its entirety.

The present disclosure encompasses the preparation and use of salts of the USP1 inhibitors, including non-toxic pharmaceutically acceptable salts. Examples of pharmaceutically acceptable addition salts include inorganic and organic acid addition salts and basic salts. Pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparaginate, glutamate and the like. The term “pharmaceutically acceptable salt” as used herein, refers to any salt, e.g., obtained by reaction with an acid or a base, of a USP1 inhibitor of the disclosure that is physiologically tolerated in the target patient (e.g., a mammal, e.g., a human).

Methods of synthesizing USP inhibitors, including c Formula I, Formula II, and Formula III are described in U.S. Publication No. 2021/0115049, which is herein incorporated by reference in its entirely. Methods of making salts and polymorphic forms of USP inhibitors, including Formula I, Formula II, and Formula III are described in PCT Publication No. WO2022/094096, which is herein incorporated by reference in its entirely.

In various aspects, the USP1 inhibitors reduce the level of USP1 protein and/or inhibit or reduce at least one biological activity of USP1 protein.

In some aspects, the USP1 inhibitors specifically bind to USP1 protein. In some aspects, the USP1 inhibitors specifically bind to USP1 protein in a USP1-UAF1 complex. In some aspects, the USP1 inhibitors specifically bind to USP1 mRNA. In some aspects, the USP1 inhibitors specifically bind to USP1 protein (alone or in a USP1-UAF1 complex) or USP1 mRNA. In some aspects, the USP1 inhibitors specifically bind to UAF1 (alone or in a USP1-UAF1 complex) and inhibit or reduces formation or activity of the USP1-UAF1 complex.

In some aspects, the USP1 inhibitors decrease the formation of the USP1-UAF1 complex. In some aspects, the USP1 inhibitors decrease the activity of the USP1-UAF1 complex. In some aspects, the USP1 inhibitors decrease the deubiquitinase activity of USP1. In some aspects, the USP1 inhibitors increase mono-ubiquitinated PCNA. In some aspects, the USP1 inhibitors increase mono-ubiquitinated FANCD2. In some aspects, the USP1 inhibitors increase mono-ubiquitinated FANCI.

In some aspects, the USP1 inhibitors do not bind to other deubiquitinases, other USP proteins, or other UAF1 complexes (e.g., USP46-UAF1) or bind deubiquitinases, other USP proteins, or other UAF1 complexes (e.g., USP46-UAF1) with at least 5-fold, at least 10-fold, at least 20-fold, or at least 100-fold reduced affinity compared to the affinity for USP1 (i.e., the K_{D} of the USP1 inhibitor for other deubiquitinases, other USP proteins, or other UAF1 complexes (e.g., USP46-UAF1) is at least 5-fold, at least 10-fold, at least 20-fold, or at least 100-fold higher than the K_{D} for USP1).

In some aspects, the USP1 inhibitors inhibit USP1 deubiquitinase activity with an IC50 of less than about 50 nM, between about 50 nM and about 200 nM, between about 200 nM and about 2 pM, or greater than 2 pM, e.g., as measured using the assay disclosed in U.S. Patent Application Publication No. 2017/0145012 or IC50 of 50 nM to 1000 nM, e.g., as measured using the assay disclosed in Liang et al., Nat Chem Biol 10:289-304 (2014). In some aspects, the USP1 inhibitors inhibit USP1 deubiquitinase activity with an IC50 as measured using the assay disclosed in Chen, et al., Chem Biol., 18 (11): 1390-1400 (2011). In some aspects, the USP1 inhibitors do not inhibit the activity of other deubiquitinases, other USP proteins, or other UAF1 complexes (e.g., USP46-UAF1) or inhibit the activity of other deubiquitinases, other USP proteins, or other UAF1 complexes (e.g., USP46-UAF1) with at least 5-fold, at least 10-fold, at least 20-fold, or at least 100-fold higher IC50 compared to the IC50 for inhibition of USP1 deubiquitinase activity.

In some aspects, the USP1 inhibitors of the present disclosure bind to a USP1 protein with an affinity in the range of 1 pM to 100 μM, or 1 pM to 1 μM, or 1 pM to 500 nM, or 1 pM to 100 nM. In some aspects, the USP1 inhibitors of the present disclosure bind to a USP1 protein with an affinity of about 1 pM to about 100 μM, about 1 nM to about 100 μM, about 1 μM to about 100 μM, about 1 μM to about 50 μM, about 1 μM to about 40 μM, about 1 μM to about 30 μM, about 1 μM to about 20 μM, or about 1 μM to about 10 μM, about 1 μM, about 5 μM, about 10 μM, about 15 μM, about 20 μM, about 25 μM, about 30 μM, about 35 μM, about 40 μM, about 45 μM, about 50 μM, about 60 μM, about 70 μM, about 80 μM, about 90 μM, or about 100 μM. In some aspects, the USP1 inhibitors of the present disclosure bind to a USP1 protein with an affinity of about 100 nM to about 1 μM, about 100 nM to about 900 nM, about 100 nM to about 800 nM, about 100 nM to about 700 nM, about 100 nM to about 600 nM, about 100 nM to about 500 nM, about 100 nM to about 400 nM, about 100 nM to about 300 nM, about 100 nM to about 200 nM, about 200 nM to about 1 μM, about 300 nM to about 1 μM, about 400 nM to about 1 μM, about 500 nM to about 1 μM, about 600 nM to about 1 μM, about 700 nM to about 1 μM, about 800 nM to about 1 μM, about 900 nM to about 1 μM, about 100 nM, about 200 nM, about 300 nM, about 400 nM, about 500 nM, about 600 nM, about 700 nM, about 800 nM, or about 900 nM. In some aspects, the USP1 inhibitors of the present disclosure bind to a USP1 protein with an affinity of about 1 nM to about 100 nM, 1 nM to about 90 nM, 1 nM to about 80 nM, 1 nM to about 70 nM, 1 nM to about 60 nM, 1 nM to about 50 nM, 1 nM to about 40 nM, 1 nM to about 30 nM, 1 nM to about 20 nM, 1 nM to about 10 nM, about 10 nM to about 100 nM, about 20 nM to about 100 nM, about 30 nM to about 100 nM, about 40 nM to about 100 nM, about 50 nM to about 100 nM, about 60 nM to about 100 nM, about 70 nM to about 100 nM, about 80 nM to about 100 nM, about 90 nM to about 100 nM, about 1 nM, about 2 nM, about 3 nM, about 4 nM, about 5 nM, about 6 nM, about 7 nM, about 8 nM, about 9 nM, about 10 nM, about 20 nM, about 30 nM, about 40 nM, about 50 nM, about 60 nM, about 70 nM, about 80 nM, about 90 nM, or about 100 nM.

In some aspects, the USP1 inhibitors of the present disclosure bind to a USP1 protein with an affinity of less than 1 μM, less than 500 nM, less than 100 nM, less than 10 nM, or less than 1 nM. In some aspects, the USP1 inhibitors bind to a USP1 protein with an affinity of less than 1 nM.

In some aspects, the USP1 inhibitors of the present disclosure inhibit USP1 activity with an IC₅₀ of 1 pM to 100 μM, or 1 pM to 1 μM, or 1 pM to 500 nM, or 1 pM to 100 nM. In some aspects, the USP1 inhibitors inhibit USP1 activity with an IC₅₀ of about 1 pM to about 100 μM, about 1 nM to about 100 μM, about 1 μM to about 100 μM, about 1 μM to about 50 μM, about 1 μM to about 40 μM, about 1 μM to about 30 μM, about 1 μM to about 20 μM, or about 1 μM to about 10 μM, about 1 μM, about 5 μM, about 10 μM, about 15 μM, about 20 μM, about 25 μM, about 30 μM, about 35 μM, about 40 μM, about 45 μM, about 50 μM, about 60 μM, about 70 μM, about 80 μM, about 90 μM, or about 100 μM. In some aspects, the USP1 inhibitors inhibit USP1 activity with an IC₅₀ of about 100 nM to about 1 μM, about 100 nM to about 900 nM, about 100 nM to about 800 nM, about 100 nM to about 700 nM, about 100 nM to about 600 nM, about 100 nM to about 500 nM, about 100 nM to about 400 nM, about 100 nM to about 300 nM, about 100 nM to about 200 nM, about 200 nM to about 1 μM, about 300 nM to about 1 μM, about 400 nM to about 1 μM, about 500 nM to about 1 μM, about 600 nM to about 1 μM, about 700 nM to about 1 μM, about 800 nM to about 1 μM, about 900 nM to about 1 μM, about 100 nM, about 200 nM, about 300 nM, about 400 nM, about 500 nM, about 600 nM, about 700 nM, about 800 nM, or about 900 nM.

In some aspects, the USP1 inhibitors of the present disclosure inhibit USP1 activity with an IC₅₀ of about 1 nM to about 100 nM, 1 nM to about 90 nM, 1 nM to about 80 nM, 1 nM to about 70 nM, 1 nM to about 60 nM, 1 nM to about 50 nM, 1 nM to about 40 nM, 1 nM to about 30 nM, 1 nM to about 20 nM, 1 nM to about 10 nM, about 10 nM to about 100 nM, about 20 nM to about 100 nM, about 30 nM to about 100 nM, about 40 nM to about 100 nM, about 50 nM to about 100 nM, about 60 nM to about 100 nM, about 70 nM to about 100 nM, about 80 nM to about 100 nM, about 90 nM to about 100 nM, about 1 nM, about 2 nM, about 3 nM, about 4 nM, about 5 nM, about 6 nM, about 7 nM, about 8 nM, about 9 nM, about 10 nM, about 20 nM, about 30 nM, about 40 nM, about 50 nM, about 60 nM, about 70 nM, about 80 nM, about 90 nM, or about 100 nM. In some aspects, the USP1 inhibitors inhibit USP1 activity with an IC₅₀ of less than 1 μM, less than 500 nM, less than 100 nM, less than 10 nM, or less than 1 nM. In some aspects, the USP1 inhibitors inhibit USP1 activity with an IC₅₀ of less than 1 nM.

Other exemplary USP1 inhibitors are disclosed, for example, in WO 2020/132269 and U.S. Provisional Application 62/857,986, each of which is herein incorporated by reference in its entirety.

### Exemplary Assays for Inhibition of USP1

Any suitable assay in the art can be used to determine an activity, detect an outcome or effect, or determine efficacy. See, e.g. U.S. Publication No. 2021/0115049, which is herein incorporated by reference in its entirely.

In some instances, a method of determining whether a USP1 inhibitor compound inhibits USP1 deubiquitinase activity measures a change in mass upon di-ubiquitin cleavage of deubiquitinase binding. For example, ubiquitin aldehyde and ubiquitin vinyl sulfone form covalent irreversible linkages to deubiquitinases that result in observable mass changes to the deubiquitinases. Similarly, cleavage of di-ubiquitins results in an observable mass change.

In some instances, a method of determining whether a USP1 inhibitor compound inhibits USP1 deubiquitinase activity involves an increase in luminescence or fluorescence upon cleavage, e.g., that can be monitored on a plate reader. Such assays can use ubiquitin linked to a fluorophore through a linker linkage, such as ubiquitin-7-amino-4-methylcoumarin (Ub-AMC) or ubiquitin-Rhodamine110. Such assays can also use a di-ubiquitin containing an isopeptide linkage. Exemplary di-ubiquitins can comprise a fluorophore on one ubiquitin and a quencher on the other ubiquitin such that fluorescence increases with then di-ubiquitin is cleaved. Such assays can also use enzyme-coupled systems wherein ubiquitin is coupled to an enzyme that is only active in producing a fluorescence enzyme product when released from the ubiquitin.

### PARP1-Selective Inhibitors

In various aspects, the PARP1-selective inhibitors of the disclosure reduce the level of PARP1 protein and/or inhibit or reduce at least one biological activity of PARP1 protein.

AZD5305 (5-[4-[(7-ethyl-6-oxo-5H-1,5-naphthyridin-3-yl)methyl]piperazin-1-yl]-N-methylpyridine-2-carboxamide) is a small molecule drug that acts by selectively inhibiting and trapping PARP1 at the sites of DNA single cell breaks (SSB). This both prevents the DNA repair and, during DNA replication, leads to the generation of the more deleterious DNA double strand breaks (DSB), when the DNA replication machinery collides with the PARP1-DNA non-covalent complexes. In contexts where the accurate DNA repair pathways are effective, such as in cells with a proficient homologous recombination repair (HRR), DSBs are accurately repaired. In contrast, in cells with deficiencies in the repair pathways, such as those with deleterious mutation in the BRCA genes, AZD5305 treatments lead to selective accumulation of genome instability which ultimately selectively kill cancer cells, while sparing normal cells. See PCT Published Appl. No. WO2022/074124, herein incorporated by reference.

The term “AZ05305” refers to a compound with the chemical name of 5-[4-[(7-ethyl-6-oxo-5H-1,5-naphthyridin-3-yl)methyl] piperazin-1-yl]-N-methyl-pyridine-2-carboxamide and structure shown below:

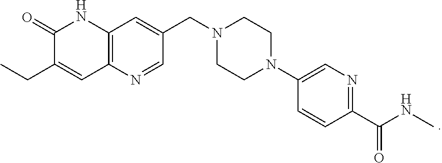 [SMILES: CCc1cc2ncc(CN3CCN(c4ccc(C(=O)NC)nc4)CC3)cc2[nH]c1=O]

PCT Published Appl. No. WO2022/074124 discloses the preparation of AZD5305. In some aspects, a free base of AZD5305, is administered to a subject. In some aspects, a pharmaceutically acceptable salt of AZD5305 is administered to a subject. In some aspects, a crystalline AZD5305 is administered to a subject. In some aspects, crystalline Form A AZD5305 is administered to a subject.

In one aspect, the PARP1-selective inhibitors are used in anti-cancer combination therapies with USP1 inhibitors of the present disclosure. In addition to the PARP1-selective inhibitor and USP1 inhibitor, other therapies can be used either before, during or after the combination therapy.

### Exemplary Assays for Inhibition of PARP

The present disclosure provides compounds that are active in inhibiting the activity of PARP1. Any suitable assay in the art can be used to determine an activity, detect an outcome or effect, or determine efficacy. See, e.g., Dillon, et al., JBS., 8 (3), 347-352 (2003); U.S. Pat. No. 9,566,276. In some aspects, a PARP1-selective inhibitor selectively inhibits and traps PARP1 in cells, e.g., as determined using the PARP trapping assay as disclosed in Illuzzi G., et al., Clinical Cancer Research, doi: 10.1158/1078-0432.CCR-22-0301 (2022). In some aspects, a PARP1-selective inhibitor does not efficiently trap PARP1 in cells.

In some aspects, the PARP1-selective inhibitors of the disclosure inhibit PARP1 activity with an IC₅₀ of less than about 50 nM, between about 50 nM and about 200 nM, between about 200 nM and about 2 pM, or greater than 2 pM.

In some aspects, the PARP1-selective inhibitors of the disclosure bind to a PARP1 protein with an affinity in the range of 1 pM to 100 μM, or 1 pM to 1 μM, or 1 pM to 500 nM, or 1 pM to 100 nM. In some aspects, the PARP1-selective inhibitors of the disclosure bind to a PARP1 protein with an affinity of about 1 pM to about 100 μM, about 1 nM to about 100 μM, about 1 μM to about 100 μM, about 1 μM to about 50 μM, about 1 μM to about 40 μM, about 1 μM to about 30 μM, about 1 μM to about 20 μM, or about 1 μM to about 10 μM, about 1 μM, about 5 μM, about 10 μM, about 15 μM, about 20 μM, about 25 μM, about 30 μM, about 35 μM, about 40 μM, about 45 μM, about 50 uM, about 60 μM, about 70 μM, about 80 μM, about 90 μM, or about 100 μM. In some aspects, the PARP1-selective inhibitors of the disclosure bind to a PARP1 protein with an affinity of about 100 nM to about 1 μM, about 100 nM to about 900 nM, about 100 nM to about 800 nM, about 100 nM to about 700 nM, about 100 nM to about 600 nM, about 100 nM to about 500 nM, about 100 nM to about 400 nM, about 100 nM to about 300 nM, about 100 nM to about 200 nM, about 200 nM to about 1 μM, about 300 M to about 1 μM, about 400 nM to about 1 μM, about 500 nM to about 1 μM, about 600 nM to about 1 μM, about 700 nM to about 1 μM, about 800 nM to about 1 μM, about 900 nM to about 1 μM, about 100 nM, about 200 nM, about 300 nM, about 400 nM, about 500 nM, about 600 nM, about 700 nM, about 800 nM, or about 900 nM. In some aspects, the PARP1-selective inhibitors of the disclosure bind to a PARP1 protein with an affinity of about 1 nM to about 100 nM, 1 nM to about 90 nM, 1 nM to about 80 nM, 1 nM to about 70 nM, 1 nM to about 60 nM, 1 nM to about 50 nM, 1 nM to about 40 nM, 1 nM to about 30 nM, 1 nM to about 20 nM, 1 nM to about 10 nM, about 10 nM to about 100 nM, about 20 nM to about 100 nM, about 30 nM to about 100 nM, about 40 nM to about 100 nM, about 50 nM to about 100 nM, about 60 nM to about 100 nM, about 70 nM to about 100 nM, about 80 nM to about 100 nM, about 90 nM to about 100 nM, about 1 nM, about 2 nM, about 3 nM, about 4 nM, about 5 nM, about 6 nM, about 7 nM, about 8 nM, about 9 nM, about 10 nM, about 20 nM, about 30 nM, about 40 nM, about 50 nM, about 60 nM, about 70 nM, about 80 nM, about 90 nM, or about 100 nM. In some aspects, the PARP1-selective inhibitors of the disclosure bind to a PARP1 protein with an affinity of less than 1 μM, less than 500 nM, less than 100 nM, less than 10 nM, or less than 1 nM. In some aspects, the PARP1-selective inhibitors of the disclosure bind to a PARP1 protein with an affinity of less than 1 nM.

In some aspects, the PARP1-selective inhibitors of the disclosure inhibit PARP1 activity with an IC₀ of 1 pM to 100 μM, or 1 pM to 1 μM, or 1 pM to 500 nM, or 1 pM to 100 nM. In some aspects, the PARP1-selective inhibitors of the disclosure inhibit PARP1 activity with an IC₅₀ of about 1 pM to about 100 μM, about 1 nM to about 100 μM, about 1 μM to about 100 μM, about 1 μM to about 50 μM, about 1 μM to about 40 μM, about 1 μM to about 30 μM, about 1 μM to about 20 μM, or about 1 μM to about 10 μM, about 1 μM, about 5 μM, about 10 μM, about 15 μM, about 20 μM, about 25 μM, about 30 μM, about 35μ, about 40μ, about 45μ, about 50μ, about 60μ, about 70 μM, about 80 μM, about 90 μM, or about 100 μM. In some aspects, the PARP1-selective inhibitors of the disclosure inhibit PARP1 activity with an IC₅₀ of about 100 nM to about 1 μM, about 100 nM to about 900 nM, about 100 nM to about 800 nM, about 100 nM to about 700 nM, about 100 nM to about 600 nM, about 100 nM to about 500 nM, about 100 nM to about 400 nM, about 100 nM to about 300 nM, about 100 nM to about 200 nM, about 200 nM to about 1 μM, about 300 nM to about 1 μM, about 400 nM to about 1 μM, about 500 nM to about 1 μM, about 600 nM to about 1 μM, about 700 nM to about 1 μM, about 800 nM to about 1 μM, about 900 nM to about 1 μM, about 100 nM, about 200 nM, about 300 nM, about 400 nM, about 500 nM, about 600 nM, about 700 nM, about 800 nM, or about 900 nM. In some aspects, the PARP1-selective inhibitors of the disclosure inhibit PARP1 activity with an IC₅₀ of about 1 nM to about 100 nM, 1 nM to about 90 nM, 1 nM to about 80 nM, 1 nM to about 70 nM, 1 nM to about 60 nM, 1 nM to about 50 nM, 1 nM to about 40 nM, 1 nM to about 30 nM, 1 nM to about 20 nM, 1 nM to about 10 nM, about 10 nM to about 100 nM, about 20 nM to about 100 nM, about 30 nM to about 100 nM, about 40 nM to about 100 nM, about 50 nM to about 100 nM, about 60 nM to about 100 nM, about 70 nM to about 100 nM, about 80 nM to about 100 nM, about 90 nM to about 100 nM, about 1 nM, about 2 nM, about 3 nM, about 4 nM, about 5 nM, about 6 nM, about 7 nM, about 8 nM, about 9 nM, about 10 nM, about 20 nM, about 30 nM, about 40 nM, about 50 nM, about 60 nM, about 70 nM, about 80 nM, about 90 nM, or about 100 nM. In some aspects, the PARP1-selective inhibitors of the disclosure inhibit PARP1 activity with an IC₅₀ of less than 1 μM, less than 500 nM, less than 100 nM, less than 10 nM, or less than 1 nM. In some aspects, the PARP1-selective inhibitors of the disclosure inhibit PARP1 activity with an IC₅₀ of less than 1 nM.

In some aspects, the PARP1-selective inhibitors have at least 500-fold selectivity for PARP1 over PARP2.

### Methods of Use

Since combinations of the disclosure are inhibitors of USP1 proteins and PARP1 proteins, the present disclosure provides a method for inhibiting a USP1 protein and/or a PARP1 protein comprising contacting a USP1 and/or PARP1 protein or a composition comprising a USP1 and/or PARP1 protein with one or more combinations of the disclosure.

Since combinations of the disclosure are inhibitors of USP1 and PARP1 proteins, a number of diseases, conditions, or disorders mediated by USP1 and/or PARP1 proteins can be treated by employing these compounds. The present disclosure is thus directed generally to a method for treating a disease, condition, or disorder responsive to the inhibition of USP1 and/or PARP1 proteins in an animal suffering from, or at risk of suffering from, the disorder, the method comprising administering to the animal an effective amount of one or more combinations of the disclosure.

The present disclosure is further directed to a method of inhibiting USP1 and/or PARP1 proteins in an animal in need thereof, the method comprising administering to the animal a therapeutically effective amount of a combination of the disclosure.

In some aspects, the combinations of the disclosure can be used to inhibit the activity of a USP1 and/or PARP1 protein. For example, in some aspects, a method of inhibiting a USP1 and/or PARP1 protein comprises contacting the USP1 and/or PARP1 protein with a combination of the disclosure. The contacting can occur in vitro or in vivo.

In some aspects, the combinations of the disclosure can be used to treat a USP1 and/or PARP1 protein mediated disorder. A USP1 and/or PARP1 protein mediated disorder is any pathological condition in which a USP1 and/or PARP1 protein is known to play a role. In some aspects, a USP1 and/or PARP1 mediated disorder is a proliferative disease such as cancer. In some aspects, the combinations of the disclosure can delay, reduce, or prevent rebounding (rapid re-growth) of a tumor.

In some aspects, the combination of the disclosure is less toxic than the PARP1-selective inhibitor alone (e.g., due to less PARP1-selective inhibitor being administered).

Various methods of treating diseases and disorders with the combinations of the disclosure are provided herein. Exemplary diseases and disorders that may be treated with the combinations of the disclosure include, but are not limited to, cancer.

In some aspects, methods of treating cancer with combinations of the disclosure are provided. Such methods comprise administering to a subject with cancer a therapeutically effective amount of a combination of the disclosure.

In some aspects, the cancer to be treated with a combination of the disclosure is selected from a hematological cancer, a lymphatic cancer, and a DNA damage repair pathway deficient cancer. In some aspects, the cancer to be treated with a combination of the disclosure is a cancer that comprises cancer cells with a mutation in a gene encoding p53. In some aspects, the cancer to be treated with a combination of the disclosure is a cancer that comprises cancer cells with a loss of function mutation in a gene encoding p53. In some aspects, the cancer to be treated with a combination of the disclosure is a cancer that comprises cancer cells with a mutation in a gene encoding BRCA1. In some aspects, the cancer to be treated with a combination of the disclosure is a cancer that comprises cancer cells with a mutation in a gene encoding BRCA2. In some aspects, the cancer to be treated with a combination of the disclosure is a cancer that comprises cancer cells with a loss of function mutation in a gene encoding ATM.

In some aspects, the cancer to be treated with a combination of the disclosure is an advanced solid tumor. An advanced solid tumor is a solid tumor that is unresectable and/or metastatic. In some aspects, the cancer to be treated with a combination of the disclosure is a solid tumor that is a resectable tumor, either benign or metastatic, where the combination is administered before and/or after a resection procedure.

In some aspects, the cancer to be treated with a combination of the disclosure is selected from non-small cell lung cancer (NSCLC), osteosarcoma, ovarian cancer, and breast cancer. In some aspects, the cancer is uterine cancer. In some aspects, the cancer is peritoneal cancer. In some aspects, the cancer is endometrial cancer, In some aspects, the cancer is ovarian cancer or breast cancer. In some aspects, the cancer is ovarian cancer. In some aspects, the cancer is breast cancer. In some aspects, the cancer is a triple negative breast cancer. In some aspects, the cancer is an ovarian cancer. In some aspects, the ovarian cancer is a BRCA1 mutant cancer, a BRCA2 mutant cancer, or a p53 mutant cancer. In some aspects, the ovarian cancer is a BRCA1 mutant cancer and a p53 mutant cancer. In some aspects, the ovarian cancer is a BRCA1 and BRCA2 mutant cancer. In some aspects, the ovarian cancer is a BRCA2 mutant cancer.

In some aspects, the cancer to be treated with a combination of the disclosure is selected from the group consisting of bone cancer, including osteosarcoma and chondrosarcoma; brain cancer, including glioma, glioblastoma, astrocytoma, medulloblastoma, and meningioma; soft tissue cancer, including rhabdoid and sarcoma; kidney cancer; bladder cancer; skin cancer, including melanoma; and lung cancer, including non-small cell lung cancer; colon cancer, uterine cancer; nervous system cancer; head and neck cancer; pancreatic cancer; and cervical cancer. In some aspects, the cancer to be treated with a combination of the disclosure is selected from the group consisting of uterine cancer, peritoneal cancer, and endometrial cancer.

Various methods of treating cancer with a combination of the disclosure are provided herein. In some aspects, a therapeutically effective amount of a combination of the disclosure is administered to a subject with cancer.

In some aspects, such methods comprise administering to a subject with triple negative breast cancer a therapeutically effective amount of a combination of the disclosure.

In some aspects, a combination of the disclosure is used to treat a cancer, wherein the cancer is a homologous-recombination deficient cancer. In some aspects, a combination of the disclosure is used to treat a cancer, wherein the cancer comprises cancer cells with a mutation in a gene encoding p53. In some aspects, a combination of the disclosure is used to treat a cancer, wherein the cancer comprises cancer cells with a loss of function mutation in a gene encoding p53. In some aspects, a combination of the disclosure is used to treat a cancer that does not have a defect in the homologous recombination pathway.

In some aspects, a combination of the disclosure is used to treat a cancer, wherein the cancer is a BRCA1 mutant cancer. In some aspects, a combination of the disclosure is used to treat a cancer, wherein the cancer is a BRCA2 mutant cancer. In some aspects, a combination of the disclosure is used to treat a cancer, wherein the cancer is a BRCA1 mutant cancer and a BRCA2 mutant cancer. In some aspects, the cancer is not a BRCA1 mutant cancer or a BRCA2 mutant cancer. In some aspects, the cancer is a BRCA1 deficient cancer. In some aspects, the cancer is a BRCA2 deficient cancer. In some aspects, the cancer is a BRCA1 deficient cancer and a BRCA2 mutant cancer.

In some aspects, a combination of the disclosure is used to treat a cancer, wherein the cancer is an ATM mutant cancer. In some aspects, the cancer is not an ATM mutant cancer. In some aspects, the cancer is an ATM deficient cancer.

In some aspects, a combination of the disclosure is used to treat a cancer, wherein the cancer is a PARP inhibitor resistant or refractory cancer. In some aspects, a combination of the disclosure is used to treat a cancer, wherein the cancer is a PARP inhibitor resistant or refractory BRCA1-deficient cancer. In some aspects, the cancer is an Olaparib-resistant or refractory cancer. In some aspects, the cancer is an AZD-5305-resistant or refractory cancer.

In some aspects, the cancer is a BRCA1 and/or BRCA2 mutant cancer, wherein the cancer comprises cells with elevated levels of RAD18, e.g., wherein the elevated levels of RAD18 are at least as high as the RAD18 protein and/or mRNA levels in ES2 cells (ES2 cells are publicly available, for example from the American Type Culture Collection (ATCC; CRL-1978)) or wherein the elevated levels of RAD18 are higher than the RAD18 protein and/or mRNA levels in HEP3B217 cells (HEP3B217 cells are publicly available, for example, from the ATCC (HB-8064)). In some aspects, a triple negative breast cancer is a BRCA1 and/or BRCA2 mutant cancer.

In some instances, the methods described herein comprise detecting RAD51 (e.g., RAD51 protein, RAD51 protein foci, and/or RAD51 mRNA) levels in cancer cells (e.g., using a sample obtained from the cancer). RAD51 protein levels can be detected using, for example, immunofluorescence, western blots, fluorescence-activated cell sorting (FACS), and/or immunohistochemistry. RAD51 mRNA levels can be detected, for example, using quantitative reverse transcriptase (RT)-polymerase chain reaction (PCR).

In some aspects, a combination of the disclosure is used to treat a cancer that was previously treated with a platinum therapy. In some aspects, a combination of the disclosure is used to treat a platinum resistant cancer. In some aspects, a combination of the disclosure is used to treat a platinum refractory cancer. In some aspects, the platinum therapy is selected from the group consisting of cisplatin, carboplatin, oxaliplatin, and combinations thereof.

In some instances, the cancer is a solid cancer. In some instances, the cancer is a hematological/lymphatic cancer. In some instances, the cancer is a DNA damage repair pathway deficient cancer. In some instances, the cancer is a homologous-recombination deficient cancer. In some instances, the cancer comprises cancer cells with a mutation in a gene encoding p53. In some instances, the cancer comprises cancer cells with a loss of function mutation in a gene encoding p53. In some instances, the cancer is selected from the group consisting of non-small cell lung cancer (NSCLC), osteosarcoma, ovarian cancer, and breast cancer (including triple negative breast cancer). In some instances, the cancer is ovarian cancer or breast cancer (including triple negative breast cancer). In some instances, the cancer is ovarian cancer. In some instances, the cancer is breast cancer (including triple negative breast cancer.) In some instances, the cancer is uterine cancer. In some instances, the cancer is peritoneal cancer. In some instances, the cancer is endometrial cancer.

In some aspects, a combination of the disclosure is used in combination with one or more additional therapeutic agents to treat cancer.

In some aspects, provided herein are combinations of the disclosure for use as a medicament or for use in preparing a medicament, e.g., for the treatment of cancer. In some aspects, provided herein are combinations of the disclosure for use in a method for the treatment of cancer.

### Pharmaceutical Combination Compositions

Combinations of the disclosure can be administered to a mammal in the form of a raw chemicals without any other components present, or combinations of the disclosure can also be administered to a mammal as part of a pharmaceutical composition containing the compound combined with a suitable pharmaceutically acceptable carrier (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Such a carrier can be selected from pharmaceutically acceptable excipients and auxiliaries. The term “pharmaceutically acceptable carrier” or “pharmaceutically acceptable vehicle” encompasses any of the standard pharmaceutical carriers, solvents, surfactants, or vehicles. Standard pharmaceutical carriers and their formulations are described in Remington's Pharmaceutical Sciences, Mack Publishing Co., Easton, PA, 19th ed. 1995. The USP1 inhibitor and the PARP1-selective inhibitor can be present in the same pharmaceutical composition; or the USP1 inhibitor and the PARP1-selective inhibitor can be in separate pharmaceutical compositions that can be administered concurrently or consecutively.

A pharmaceutical combination composition of the present disclosure may be prepared as liquid suspensions or solutions using a liquid, such as an oil, water, an alcohol, and combinations of these.

As provided herein, a USP1 inhibitor (e.g., 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine or a pharmaceutically acceptable salt, solvate, hydrate, or co-crystal thereof) can be in any pharmaceutical composition including all compositions where such an inhibitor is combined with one or more pharmaceutically acceptable carriers. In some aspects, the USP1 inhibitor is present in a composition in an amount that is effective to achieve its intended therapeutic purpose.

In some aspects, the present disclosure provides a pharmaceutical composition comprising a USP1 inhibitor (e.g., 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine or a pharmaceutically acceptable salt, solvate, hydrate, or co-crystal thereof), and a pharmaceutically acceptable carrier. In some aspects, a pharmaceutical composition comprises crystalline Form 2 of the gentisic acid co-crystal of Formula I.

The pharmaceutical combination compositions to be used for in vivo administration can be sterile. This is readily accomplished by filtration through, e.g., sterile filtration membranes.

Pharmaceutical combination compositions within the scope of the present disclosure include all compositions where a USP1 inhibitor and a PARP1-selective inhibitor of the disclosure are combined with one or more pharmaceutically acceptable carriers. In one aspect, the USP1 inhibitor and PARP1-selective inhibitor of the disclosure are present in the composition in an amount that is effective to achieve its intended therapeutic purpose.

A pharmaceutical combination composition of the present disclosure can be administered to any patient that may experience the beneficial effects of a combination of the disclosure. Foremost among such patients are mammals, e.g., humans and companion animals, although the disclosure is not intended to be so limited. In one aspect, the patient is a human. In another aspect, a pharmaceutical combination composition of the present disclosure can be administered to a patient having PARP inhibitor resistant or refractory cancer. In another aspect, a pharmaceutical combination composition of the present disclosure can be administered to a patient having PARP inhibitor resistant or refractory BRCA1-deficient cancer

In another aspect, the present disclosure provides kits that comprise a combination of the disclosure packaged in a manner that facilitates their use to practice methods of the present disclosure. In one aspect, the kit includes a USP1 inhibitor and a PARP1-selective inhibitor of the disclosure packaged in a container, such as a sealed bottle or vessel, with a label affixed to the container or included in the kit that describes use of the compounds to practice the methods of the disclosure. In one aspect, the combination composition is packaged in a unit dosage form. The kit further can include a device suitable for administering the combination composition according to the intended route of administration. In some aspects, the present disclosure provides a kit that comprises a USP1 inhibitor and a PARP1-selective inhibitor of the disclosure, or a pharmaceutically acceptable salt or solvate thereof, and instructions for administering the compounds, or pharmaceutically acceptable salts or solvates thereof, to a patient having cancer.

In some aspects, the present disclosure provides a pharmaceutical combination composition comprising a USP1 inhibitor and a PARP1-selective inhibitor of the disclosure, or a pharmaceutically acceptable salt or solvate thereof, and a pharmaceutically acceptable carrier.

In some aspects, the present disclosure provides a pharmaceutical combination composition comprising a USP1 inhibitor and a PARP1-selective inhibitor of the disclosure, or a pharmaceutically acceptable salt or solvate thereof, and a pharmaceutically acceptable carrier, wherein the combination binds to a protein encoded by the USP1 gene and/or a PARP1 gene.

In some aspects, the present disclosure provides a pharmaceutical combination composition comprising a USP1 inhibitor and a PARP1-selective inhibitor of the disclosure, or a pharmaceutically acceptable salt or solvate thereof, and a pharmaceutically acceptable carrier, wherein the pharmaceutical composition is for use in treating cancer.

In some aspects, the present disclosure provides a pharmaceutical combination composition comprising a USP1 inhibitor and a PARP1-selective inhibitor of the disclosure, or a pharmaceutically acceptable salt or solvate thereof, and a pharmaceutically acceptable carrier, wherein the pharmaceutical composition is for the manufacture of a medicament for treatment of cancer.

### EXAMPLES

### Example 1: Anti-Tumor Activity of Formula I Co-Crystal in Combination with the Selective PARP1 Inhibitor, AZD5305, in a Patient-Derived Breast Xenograft Model in Nude Mice

The USP1 inhibitor employed in the examples is the co-crystal described above in paragraphs and [0143]-[0155]. This co-crystal can be prepared as described in Example 6 of Published PCT WO2022/094096. The Formula I co-crystal is a co-crystal of gentisic acid and 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine.

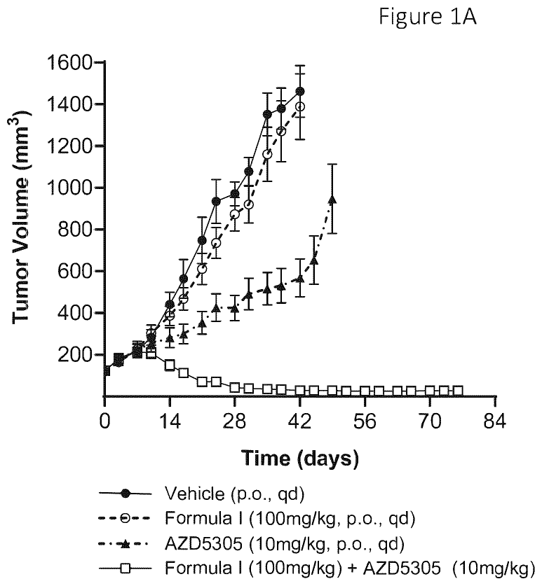

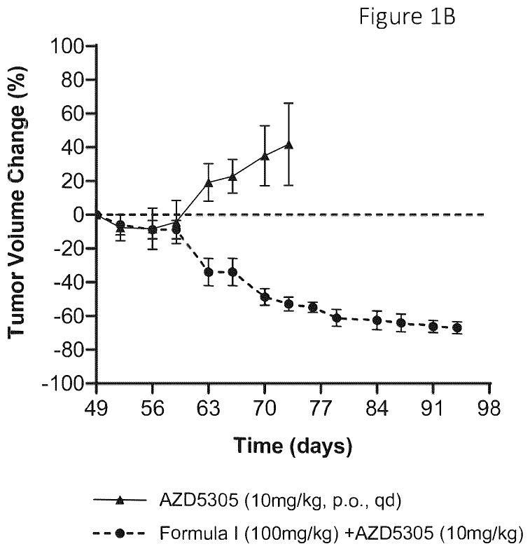

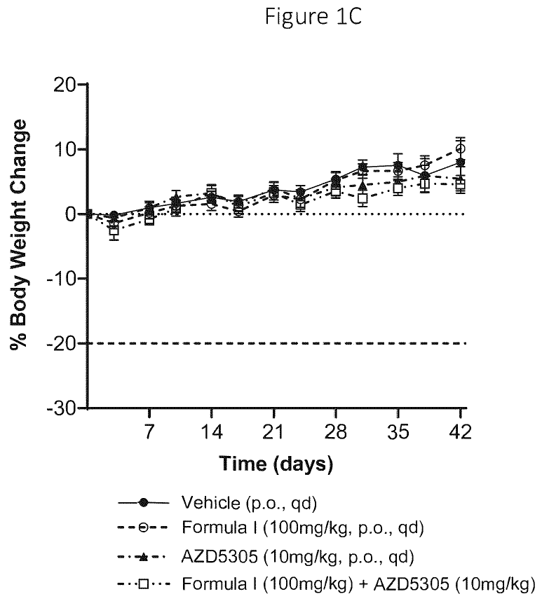

Anti-tumor activity of the USP1 inhibitor of Formula I co-crystal in combination with AZD5305 was evaluated in mice using an olaparib resistant patient-derived breast xenograft model in nude mice, as shown in FIGS. 1A through 1C. 6-9 week old female Athymic nude mice from Envigo were anesthetized, and a 20 mm³ tumor fragment was placed subcutaneously via incision in the flank. When tumors established to a tumor volume ranging from 63 to 196 mm³, mice were randomized into groups of 10 and were assigned into the following groups: control, Formula I (100 mg/kg), AZD5305 (10 mg/kg) or Formula I (100 mg/kg) and AZD5305 (10 mg/kg) in combination. Compound was administered via oral gavage once daily for at least 42 days dependent upon the treatment group. Body weight and tumor volume were measured twice per week. Tumor volume was calculated as mean and standard error of the mean for each treatment group.

The data in FIG. 1A show that, compared to equivalent doses of the single agent of Formula I or AZD5305, the combination treatment group showed enhanced anti-tumor activity in the patient-derived subcutaneous mouse model.

To evaluate combination activity in mice with tumors resistance to PARP inhibitors, on day 49 of treatment, 5 mice that demonstrated resistance to AZD5305 monotherapy were subsequently dosed with Formula I (100 mg/kg) and AZD5305 (10 mg/kg) in combination. Compared to the remaining animals treated with AZD5305 monotherapy, the addition of Formula I led to a decrease in tumor volume, as shown in FIG. 1B.

Tolerability of AZD5305 (10 mg/kg) and Formula I (100 mg/kg) in the combination efficacy study, was assessed by monitoring body weight and calculating body weight changes as % from body weight on day of treatment start (day 0), as shown in FIG. 1C.

### Example 2: In Vitro Efficacy

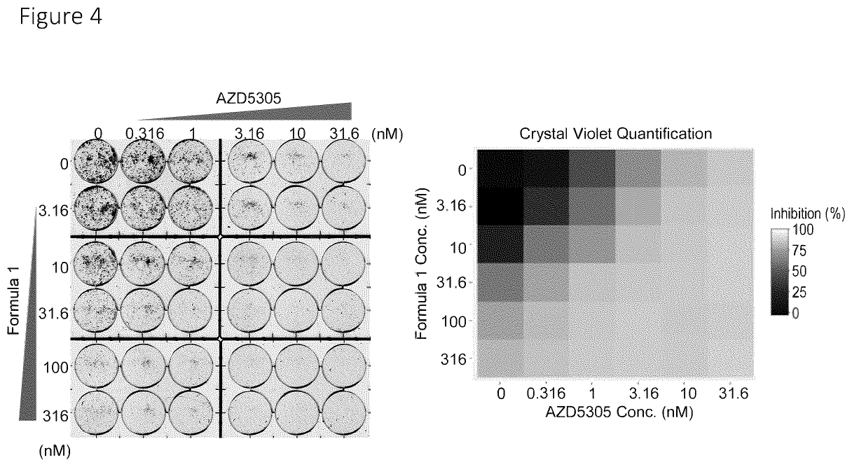

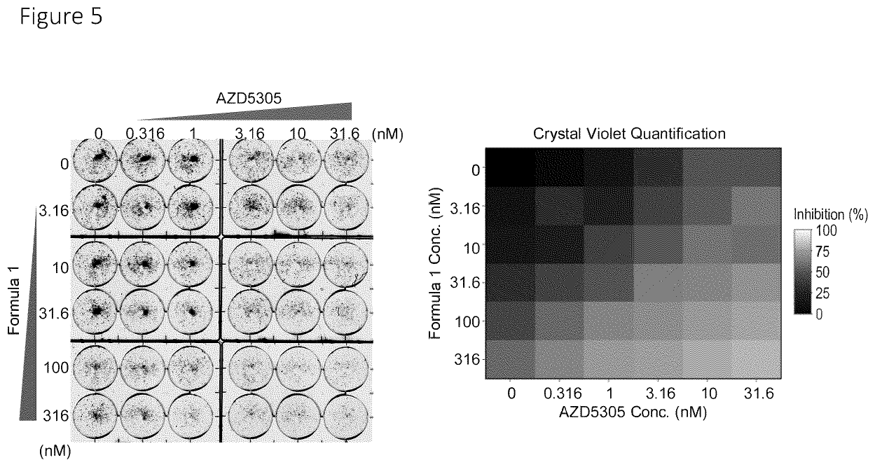

In vitro experiments were conducted using the colony formation unit (CFU) assay. UWB1.289 parental or UWB1.289 BRCA1 overexpressing cells were plated at a concentration of 2500 cells/well on day-1. On day 0, the wells were treated with DMSO or increasing concentrations of Formula I and/or AZD5305. On day 10, when clearly interspersed colonies were visible in the DMSO treated wells, the cells were fixed and stained using 0.1% crystal violet in 10% ethanol for 20 minutes at room temperature. The plates were imaged and the amount of crystal violet stain in each well was quantified by extracting the crystal violet into 10% acetic acid and measuring the absorbance at 565 nm. The CFU results are shown in FIG. 4 and FIG. 5. The results demonstrate that the USP1 inhibitor and PARP1-selective inhibitor are an effective combination in UWB1.289 and UWB1.289 BRCA1 overexpressing cells.

Having now fully described this invention, it will be understood by those of ordinary skill in the art that the same can be performed within a wide and equivalent range of conditions, formulations, and other parameters without affecting the scope of the invention or any aspect thereof.

Other aspects of the invention will be apparent to those skilled in the art from consideration of the specification and practice of the invention disclosed herein. It is intended that the specification and examples be considered as exemplary only, with a true scope and spirit of the invention being indicated by the following claims.

All patents and publications cited herein are fully incorporated by reference herein in their entirety.

1. A method of treating cancer in a subject comprising administering to the subject (i) a ubiquitin-specific-processing protease 1 (USP1) inhibitor and (ii) a poly ADP-ribose polymerase 1 (PARP1)-selective inhibitor, or a pharmaceutically acceptable salt, hydrate, solvate, amorphous solid, polymorph, or co-crystal thereof,
wherein the USP1 inhibitor is a compound selected from the group consisting of 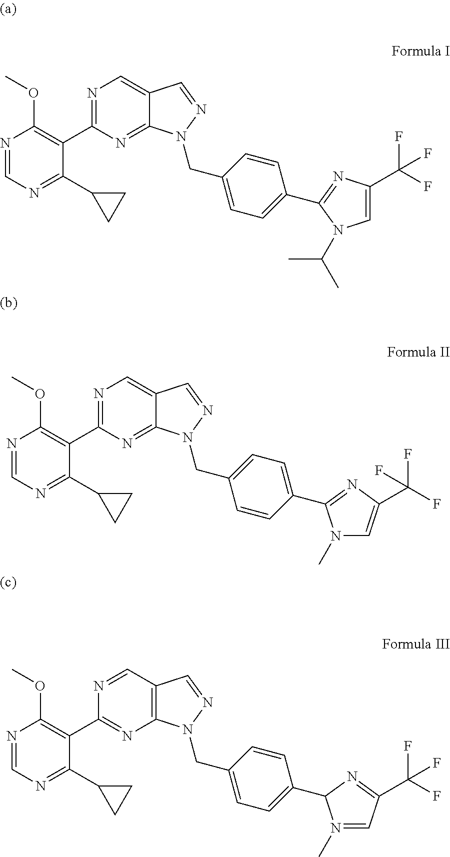
and pharmaceutically acceptable salts, hydrates, solvates, amorphous solids, polymorphs, or co-crystals, thereof.

2. A method of delaying, reducing, or preventing rebounding of a tumor in a subject comprising administering to the subject (i) a ubiquitin-specific-processing protease 1 (USP1) inhibitor and (ii) a PARP1-selective inhibitor, or a pharmaceutically acceptable salt, hydrate, solvate, amorphous solid, polymorph, or co-crystal thereof,
wherein the USP1 inhibitor is a compound selected from the group consisting of 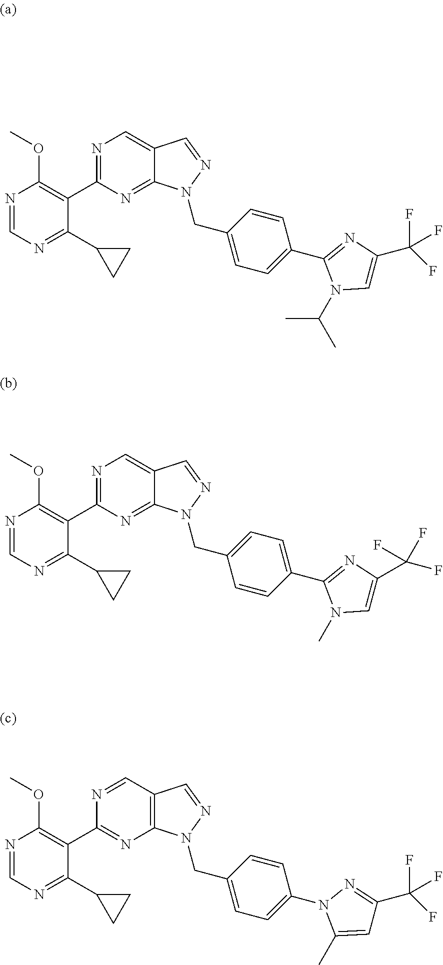 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1 ; COc1ncnc(C2CC2)c1-c1ncc2cnn(Cc3ccc(-n4nc(C(F)(F)F)cc4C)cc3)c2n1]
and pharmaceutically acceptable salts, hydrates, solvates, amorphous solids, polymorphs, or co-crystals thereof.

3. The method of claim 1, wherein said PARP1-selective inhibitor is AZD5305 or a pharmaceutically acceptable salt, hydrate, solvate, amorphous solid, or polymorph thereof.

4. The method of claim 1, wherein the USP1 inhibitor is the compound of Formula I, or a pharmaceutically acceptable salt, hydrate, solvate, amorphous solid, polymorph, or co-crystal thereof.

5. The method of claim 1, wherein the USP1 inhibitor is the compound of Formula II, or a pharmaceutically acceptable salt, hydrate, solvate, amorphous solid, polymorph, or co-crystal thereof.

6. The method of claim 1, wherein the USP1 inhibitor is the compound of Formula III, or a pharmaceutically acceptable salt, hydrate, solvate, amorphous solid, polymorph, or co-crystal thereof.

7. The method of claim 1, wherein the USP1 inhibitor is provided as a co-crystal of a pharmaceutically acceptable acid and the USP1 inhibitor.

8. The method of claim 7, wherein the USP1 inhibitor is a co-crystal of a pharmaceutically acceptable acid and 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine, optionally wherein the pharmaceutically acceptable acid is a gentisic acid, benzoic acid, or salicylic acid co-crystal.

9. The method of claim 1, wherein the administration of the USP1 inhibitor, or said pharmaceutically acceptable salt, hydrate, solvate, amorphous solid, polymorph, or co-crystals thereof, and said PARP1-selective inhibitor, or said pharmaceutically acceptable salt, hydrate, solvate, amorphous solid, polymorph, or co-crystal thereof, provides a synergistic effect.

10. The method of claim 1, wherein said USP1 inhibitor, or said pharmaceutically acceptable salt, hydrate, solvate, amorphous solid, polymorph, or co-crystals thereof, and said PARP1-selective inhibitor, or said pharmaceutically acceptable salt, hydrate, solvate, amorphous solid, polymorph, or co-crystals thereof, are administered in a therapeutically effective amount sufficient to produce one or more therapeutic effects selected from the group consisting of (i) reduction in tumor size, (ii) increase in tumor regression rate, and (iii) reduction or inhibition of tumor growth.

11. The method of claim 1, wherein said USP1 inhibitor, or said pharmaceutically acceptable salt, hydrate, solvate, amorphous solid, polymorph, or co-crystals thereof, and said PARP1-selective inhibitor, or said pharmaceutically acceptable salt, hydrate, solvate, amorphous solid, polymorph, or co-crystals thereof, delay, reduce, or prevent rebounding of a tumor.

12. The method of claim 1, wherein the USP1 inhibitor, or said pharmaceutically acceptable salt, hydrate, solvate, amorphous solid, polymorph, or co-crystals thereof, and said PARP1-selective inhibitor, or said pharmaceutically acceptable salt, hydrate, solvate, amorphous solid, polymorph, or co-crystals thereof, are administered sequentially.

13. The method of claim 1, wherein the USP1 inhibitor, or said pharmaceutically acceptable salt, hydrate, solvate, amorphous solid, polymorph, or co-crystals thereof, and the PARP1-selective inhibitor, or said pharmaceutically acceptable salt, hydrate, solvate, amorphous solid, polymorph, or co-crystals thereof, are administered simultaneously.

14. The method of claim 1, wherein the USP1 inhibitor and/or the PARP1-selective inhibitor is administered at a dose that is not effective as a single agent.

15. The method of claim 1, wherein the subject is a mammal, optionally, wherein the mammal is a human.

16. A combination composition comprising (i) a ubiquitin-specific-processing protease 1 (USP1) inhibitor and (ii) a PARP1-selective inhibitor, or a pharmaceutically acceptable salt, hydrate, solvate, amorphous solid, polymorph, or co-crystals thereof, wherein the USP1 inhibitor is a compound selected from the group consisting of 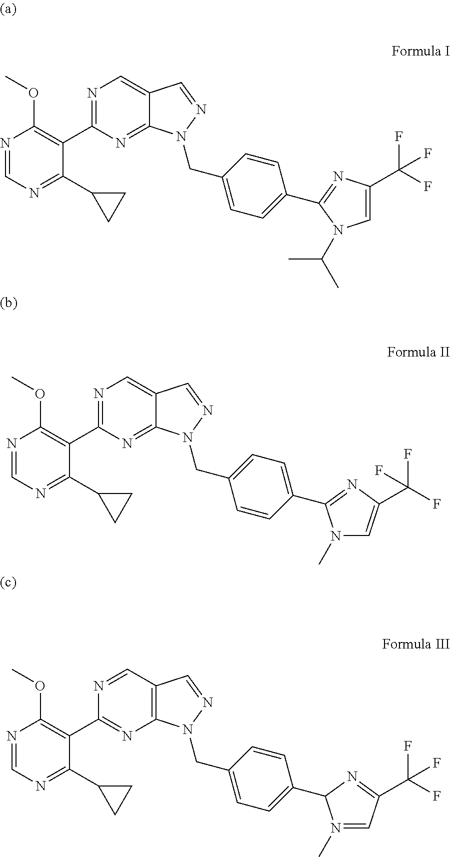
and pharmaceutically acceptable salts, hydrates, solvates, amorphous solids, or polymorphs thereof.

17. The composition of claim 16, wherein said PARP1-selective inhibitor is AZD5305 and pharmaceutically acceptable salts, hydrates, solvates, amorphous solids, polymorphs, or co-crystals thereof.

18. The composition of claim 16, wherein the USP1 inhibitor is the compound of Formula I, or a pharmaceutically acceptable salt, hydrate, solvate, amorphous solid, or polymorph thereof.

19. The composition of claim 16, wherein the USP1 inhibitor is the compound of Formula II, or a pharmaceutically acceptable salt, hydrate, solvate, amorphous solid, or polymorph thereof.

20. The composition of claim 16, wherein the USP1 inhibitor is the compound of Formula III, or a pharmaceutically acceptable salt, hydrate, solvate, amorphous solid, polymorph, or co-crystals thereof.

21-47. (canceled)
